SEQUENCE LISTING (109 sequences)

SEQ ID NO: 1 - protein, 121 aa, Artificial Sequence
  Heavy chain variable region
QVQLQESGPGLVKPSETLSLTCTVSNGSLGFDFWSWIRQPPGRGLEWIGYIYYSGSWSLNPSFKGRVTMSVDTSKNQFSLNLRSVTAADTAVYYCARGGYTGYGGDWFDPWGQGTLVTVSS

SEQ ID NO: 2 - protein, 121 aa, Artificial Sequence
  Heavy chain variable region
QVQLQESGPGLVKPSETLSLTCTVSNGSLGFEFWSWIRQPPGRGLEWIGYIVYSGSHSVSPSLKTRVTMSVDTSKNQFSLNLRSVTAADTAVYYCARGGYTGHGGDWFDTWGQGTLVTVSS

SEQ ID NO: 3 - protein, 127 aa, Artificial Sequence
  Heavy chain variable region
QVQLVQSGSELKKPGASVKVSCKASGYTFTRFALSWVRQAPGQGLEWMGWIDPNTGTPTYAQDFTGRFVFSLDTSVTTAYLQISSLKAEDTAVYYCARSLGYCGSDICYPNGILDNWGQGTLVTVSS

SEQ ID NO: 4 - protein, 127 aa, Artificial Sequence
  Heavy chain variable region
QVQLVQSGSELKKPGASVKVSCKASGYTFTRFAVNWVRQAPGQGLEWMGWIDPNTGTPTYAQGVTNRFVFSLDTSVTTAYLQISSLKAEDTAVYYCARSLGYCSSDICYPNLIFDNWGQGTLVTVSS

SEQ ID NO: 5 - protein, 127 aa, Artificial Sequence
  Heavy chain variable region
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSS

SEQ ID NO: 6 - protein, 121 aa, Artificial Sequence
  Heavy chain variable region
QVQLQESGPGLVKPSETLSLTCTVSDGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 7 - protein, 121 aa, Artificial Sequence
  Heavy chain variable region
QVQLQESGPGLVKPSETLSLTCTVSEGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 8 - protein, 127 aa, Artificial Sequence; identical to SEQ ID NO: 5
  Heavy chain variable region

SEQ ID NO: 9 - protein, 121 aa, Artificial Sequence
  Heavy chain variable region
QVQLQESGPGLVKPSETLSLTCTVSNGSLGFYFWSWIRQPPGRGLEWIGYIYYSGSTSFNPSLKSRVTMSVDTSKNQFSLNLRSVTAADTAVYYCARGGYTGYGGDWFDPWGQGTLVTVSS

SEQ ID NO: 10 - protein, 127 aa, Artificial Sequence
  Heavy chain variable region
QVQLVQSGSELKKPGASVKVSCKASGYTFTRFTMSWVRQAPGQGLEWMGWINPNTGNPTYAQDFTGRFVFSLDTSVTTAYLQISSLKAEDTAVYYCARILGYCNTDNCYPNWIFDYWGQGTLVTVSS

SEQ ID NO: 11 - protein, 122 aa, Artificial Sequence
  Heavy chain variable region
QVQLQESGPGLVRPSETLSLTCTVSGGSISSYSWSWIRQPPGKGLEWIGYIDYSGSTNYNPSLKSRVTISVDTSKTQFSLKLSSVSAADTAVYYCAKDLLYKWNYVEGFDIWGQGTTVTVSS

SEQ ID NO: 12 - protein, 118 aa, Artificial Sequence
  Heavy chain variable region
EVQLVESGGGVVQPGRSLRLSCAASGFTFSSYDTHWVRQAPGKGLEWVAVISYDGSNKYYADSVKGRFTISRDNSKNTLYLQMNSLRAEDTAMYYCARERGWDVFDIWGQGTLVTVSS

SEQ ID NO: 13 - protein, 119 aa, Artificial Sequence
  Heavy chain variable region
EVQLVESGGGVVQPGRSLRLSCAASGFTFSSYGMHWVRQAPGKGLEWVAVISYHGSDKYYADSVKGRFTISRDNSKNTLYLQMNSLRAEDTAVYYCARDGDNWDVFDIWGQGTLVTVSS

SEQ ID NO: 14 - protein, 119 aa, Artificial Sequence
  Heavy chain variable region
EVQLVQSGSELKKPGASVKVSCKASGYTFTTNALNWVRQAPGQGLEWMGWINTHTGNPTYAQGFIGRFVFSLDTSVSTAYLQIRSLKAEDTAVYYCAREPNWGVYFDYWGQGTLVTVSS

SEQ ID NO: 15 - protein, 119 aa, Artificial Sequence
  Heavy chain variable region
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARDGSGWDDFDYWGQGTLVTVSS

SEQ ID NO: 16 - protein, 121 aa, Artificial Sequence
  Heavy chain variable region
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARGSILAAQMWGDIWGQGTLVTVSS

SEQ ID NO: 17 - protein, 119 aa, Artificial Sequence
  Heavy chain variable region
QVQLVQSGSELKKPGASVKVSCKASGYTFTTNALNWVRQAPGQGLEWMGWINTHTGNPTYAQGFIGRFVFSLDTSVSTAYLQIRSLKAEDTAVYYCAREPNWGVYFDYWGQGTLVTVSS

SEQ ID NO: 18 - protein, 443 aa, Artificial Sequence
  Heavy chain nivolumab analog 1
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 440 aa, Artificial Sequence
  Heavy chain nivolumab analog 2
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 440 aa, Artificial Sequence; identical to SEQ ID NO: 19
  Heavy chain nivolumab analog 3

SEQ ID NO: 21 - protein, 440 aa, Artificial Sequence; identical to SEQ ID NO: 19
  Heavy chain nivolumab analog 4

SEQ ID NO: 22 - protein, 214 aa, Artificial Sequence
  Light chain nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 122 aa, Artificial Sequence
  Heavy chain variable region negative control
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVT

SEQ ID NO: 24 - protein, 107 aa, Artificial Sequence
  Light chain variable region negative control
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 25 - protein, 447 aa, Artificial Sequence
  Heavy chain motavizumab analog
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 213 aa, Artificial Sequence
  Light chain motavizumab analog
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 447 aa, Artificial Sequence
  Heavy chain relatlimab analog
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 214 aa, Artificial Sequence
  Light chain relatlimab analog
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 98 aa, Artificial Sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 30 - protein, 110 aa, Artificial Sequence
  CH2
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 31 - protein, 107 aa, Artificial Sequence
  CH3
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 110 aa, Artificial Sequence; identical to SEQ ID NO: 30
  CH2

SEQ ID NO: 33 - protein, 107 aa, Artificial Sequence
  CH3
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 113 aa, Artificial Sequence [sequence omitted]

SEQ ID NO: 35 - protein, 107 aa, Artificial Sequence [sequence omitted]

SEQ ID NO: 36 - protein, 5 aa, Artificial Sequence
  HCDR1 according to Kabat
FDFWS

SEQ ID NO: 37 - protein, 16 aa, Artificial Sequence
  HCDR2 according to Kabat
YIYYSGSWSLNPSFKG

SEQ ID NO: 38 - protein, 13 aa, Artificial Sequence
  HCDR3 according to Kabat
GGYTGYGGDWFDP

SEQ ID NO: 39 - protein, 5 aa, Artificial Sequence
  HCDR1 according to Kabat
FEFWS

SEQ ID NO: 40 - protein, 16 aa, Artificial Sequence
  HCDR2 according to Kabat
YIVYSGSHSVSPSLKT

SEQ ID NO: 41 - protein, 13 aa, Artificial Sequence
  HCDR3 according to Kabat
GGYTGHGGDWFDT

SEQ ID NO: 42 - protein, 5 aa, Artificial Sequence
  HCDR1  according to Kabat
RFALS

SEQ ID NO: 43 - protein, 17 aa, Artificial Sequence
  HCDR2 according to Kabat
WIDPNTGTPTYAQDFTG

SEQ ID NO: 44 - protein, 18 aa, Artificial Sequence
  HCDR3  according to Kabat
SLGYCGSDICYPNGILDN

SEQ ID NO: 45 - protein, 5 aa, Artificial Sequence
  HCDR1 according to Kabat
RFAVN

SEQ ID NO: 46 - protein, 17 aa, Artificial Sequence
  HCDR2 according to Kabat
WIDPNTGTPTYAQGVTN

SEQ ID NO: 47 - protein, 18 aa, Artificial Sequence
  HCDR3 according to Kabat
SLGYCSSDICYPNLIFDN

SEQ ID NO: 48 - protein, 5 aa, Artificial Sequence
  HCDR1 according to Kabat
RFALH

SEQ ID NO: 49 - protein, 17 aa, Artificial Sequence
  HCDR2 according to Kabat
WIDPNTGTPTFAQGVTG

SEQ ID NO: 50 - protein, 18 aa, Artificial Sequence
  HCDR3 according to Kabat
SLGYCDSDICYPNWIFDN

SEQ ID NO: 51 - protein, 5 aa, Artificial Sequence
  HCDR1 according to Kabat
YHFWS

SEQ ID NO: 52 - protein, 16 aa, Artificial Sequence
  HCDR2 according to Kabat
YIVYSGSYNVNPSLKT

SEQ ID NO: 53 - protein, 13 aa, Artificial Sequence
  HCDR3 according to Kabat
GGYTGYGGDWFDP

SEQ ID NO: 54 - protein, 5 aa, Artificial Sequence
  HCDR1 according to Kabat
YHFWS

SEQ ID NO: 55 - protein, 16 aa, Artificial Sequence
  HCDR2 according to Kabat
YIVYSGSYNVNPSLKT

SEQ ID NO: 56 - protein, 13 aa, Artificial Sequence
  HCDR3 according to Kabat
GGYTGYGGDWFDP

SEQ ID NO: 57 - protein, 5 aa, Artificial Sequence
  HCDR1 according to Kabat
RFALH

SEQ ID NO: 58 - protein, 17 aa, Artificial Sequence
  HCDR2 according to Kabat
WIDPNTGTPTFAQGVTG

SEQ ID NO: 59 - protein, 18 aa, Artificial Sequence
  HCDR3 according to Kabat
SLGYCDSDICYPNWIFDN

SEQ ID NO: 60 - protein, 6 aa, Artificial Sequence
  LCDR1 according to IMGT
QSISSY

SEQ ID NO: 61 - protein, 3 aa, Artificial Sequence
  LCDR2 according to IMGT
AAS

SEQ ID NO: 62 - protein, 9 aa, Artificial Sequence
  LCDR3 according to IMGT
QQSYSTPPT

SEQ ID NO: 63 - protein, 108 aa, Artificial Sequence
  Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 64 - protein, 107 aa, Artificial Sequence
  Light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 65 - protein, 108 aa, Artificial Sequence
  Light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 66 - protein, 108 aa, Artificial Sequence
  Light chain variable region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 67 - protein, 95 aa, Artificial Sequence
  V region
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 68 - protein, 95 aa, Artificial Sequence
  V region
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 69 - protein, 96 aa, Artificial Sequence
  V region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 70 - protein, 96 aa, Artificial Sequence
  V region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 71 - protein, 107 aa, Artificial Sequence [sequence omitted]

SEQ ID NO: 72 - protein, 110 aa, Artificial Sequence
  CH2
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAK

SEQ ID NO: 73 - protein, 107 aa, Artificial Sequence
  CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 5 aa, Artificial Sequence
  HCDR1 according to Kabat
SYSWS

SEQ ID NO: 75 - protein, 16 aa, Artificial Sequence
  HCDR2 according to Kabat
YIDYSGSTNYNPSLKS

SEQ ID NO: 76 - protein, 14 aa, Artificial Sequence
  HCDR3 according to Kabat
DLLYKWNYVEGFDI

SEQ ID NO: 77 - protein, 5 aa, Artificial Sequence
  HCDR1 according to Kabat
SYDTH

SEQ ID NO: 78 - protein, 17 aa, Artificial Sequence
  HCDR2 according to Kabat
VISYDGSNKYYADSVKG

SEQ ID NO: 79 - protein, 9 aa, Artificial Sequence
  HCDR3 according to Kabat
ERGWDVFDI

SEQ ID NO: 80 - protein, 5 aa, Artificial Sequence
  HCDR1 according to Kabat
SYGMH

SEQ ID NO: 81 - protein, 17 aa, Artificial Sequence
  HCDR2 according to Kabat
VISYHGSDKYYADSVKG

SEQ ID NO: 82 - protein, 10 aa, Artificial Sequence
  HCDR3 according to Kabat
DGDNWDVFDI

SEQ ID NO: 83 - protein, 5 aa, Artificial Sequence
  HCDR1 according to Kabat
TNALN

SEQ ID NO: 84 - protein, 17 aa, Artificial Sequence
  HCDR2 according to Kabat
WINTHTGNPTYAQGFIG

SEQ ID NO: 85 - protein, 10 aa, Artificial Sequence
  HCDR3 according to Kabat
EPNWGVYFDY

SEQ ID NO: 86 - protein, 5 aa, Artificial Sequence
  HCDR1 according to Kabat
SYGIS

SEQ ID NO: 87 - protein, 17 aa, Artificial Sequence
  HCDR2 according to Kabat
WISAYSGNTNYAQKLQG

SEQ ID NO: 88 - protein, 10 aa, Artificial Sequence
  HCDR3 according to Kabat
DGSGWDDFDY

SEQ ID NO: 89 - protein, 5 aa, Artificial Sequence
  HCDR1 according to Kabat
SYGIS

SEQ ID NO: 90 - protein, 17 aa, Artificial Sequence
  HCDR2 according to Kabat
WISAYSGNTNYAQKLQG

SEQ ID NO: 91 - protein, 12 aa, Artificial Sequence
  HCDR3 according to Kabat
GSILAAQMWGDI

SEQ ID NO: 92 - protein, 5 aa, Artificial Sequence
  HCDR1 according to Kabat
TNALN

SEQ ID NO: 93 - protein, 17 aa, Artificial Sequence
  HCDR2 according to Kabat
WINTHTGNPTYAQGFIG

SEQ ID NO: 94 - protein, 10 aa, Artificial Sequence
  HCDR3 according to Kabat
EPNWGVYFDY

SEQ ID NO: 95 - protein, 5 aa, Artificial Sequence
  HCDR1
  MISC_FEATURE (1)..(1): X can be F, Y, T, or H
  MISC_FEATURE (2)..(2): X can be Y, Q, E, H, or D
  MISC_FEATURE (4)..(4): X can be W, or Y
XXFXS

SEQ ID NO: 96 - protein, 16 aa, Artificial Sequence
  HCDR2
  MISC_FEATURE (3)..(3): X can be Y, V, or I
  MISC_FEATURE (7)..(7): X can be S, or G
  MISC_FEATURE (8)..(8): X can be T, Y, S, H, N, W, L, or Q
  MISC_FEATURE (9)..(9): X can be S, or N
  MISC_FEATURE (10)..(10): X can be F, V, or L
  MISC_FEATURE (11)..(11): X can be N, or S
  MISC_FEATURE (13)..(13): X can be S or A
  MISC_FEATURE (14)..(14): X can be F or L
  MISC_FEATURE (16)..(16): X can be S, T, G, D, R, or N
YIXYSGXXXXXPXXKX

SEQ ID NO: 97 - protein, 13 aa, Artificial Sequence
  HCDR3
  MISC_FEATURE (6)..(6): X can be Y, H, V, or A
  MISC_FEATURE (13)..(13): X can be P, V, Y, W, F, T, Q, H, or S
GGYTGXGGDWFDX

SEQ ID NO: 98 - protein, 5 aa, Artificial Sequence
  HCDR1
  MISC_FEATURE (2)..(2): X can be F, or Y
  MISC_FEATURE (3)..(3): X can be T, A, or V
  MISC_FEATURE (4)..(4): X can be M, L, or V
  MISC_FEATURE (5)..(5): X can be S, H, N, V, or T
RXXXX

SEQ ID NO: 99 - protein, 17 aa, Artificial Sequence
  HCDR2
  MISC_FEATURE (3)..(3): X can be N, or D
  MISC_FEATURE (4)..(4): X can be P, S, or T
  MISC_FEATURE (5)..(5): X can be N, or Q
  MISC_FEATURE (6)..(6): X can be T, or D
  MISC_FEATURE (8)..(8): X can be N, S, T, K, L, or E
  MISC_FEATURE (9)..(9): X can be P, Y, A, H, or F
  MISC_FEATURE (10)..(10): X can be T, or S
  MISC_FEATURE (11)..(11): X can be Y, F, or H
  MISC_FEATURE (12)..(12): X can be A, G, V, or F
  MISC_FEATURE (13)..(13): X can be Q, R, N, L, T, or S
  MISC_FEATURE (14)..(14): X can be D, A, G, or S
  MISC_FEATURE (15)..(15): X can be F, V, or A
  MISC_FEATURE (16)..(16): X can be T, K, H, G
  MISC_FEATURE (17)..(17): X can be G, N, E, or D
WIXXXXGXXXXXXXXXX

SEQ ID NO: 100 - protein, 18 aa, Artificial Sequence
  HCDR3
  MISC_FEATURE (1)..(1): X can be I, S, or V
  MISC_FEATURE (2)..(2): X can be L, Q, or N
  MISC_FEATURE (6)..(6): X can be N, G, S, or D
  MISC_FEATURE (7)..(7): X can be T, S, P, N, or E
  MISC_FEATURE (9)..(9): X can be N, or I
  MISC_FEATURE (14)..(14): X can be W, G, Q, H, W, A, or L
  MISC_FEATURE (15)..(15): X can be I, V, or L
  MISC_FEATURE (16)..(16): X can be F, L, or I
  MISC_FEATURE (18)..(18): X can be Y, S, N, I, R, H, V, T, K, A, or L
XXGYCXXDXCYPNXXXDX

SEQ ID NO: 101 - protein, 5 aa, Artificial Sequence
  HCDR1
  MISC_FEATURE (2)..(2): X can be Y or F
  MISC_FEATURE (3)..(3): X can be Y or S
SXXWS

SEQ ID NO: 102 - protein, 16 aa, Artificial Sequence
  HCDR2
  MISC_FEATURE (3)..(3): X can be Y or D
  MISC_FEATURE (7)..(7): X can be S, or T
  MISC_FEATURE (10)..(10): X can be Y or F
  MISC_FEATURE (13)..(13): X can be S, or F
  MISC_FEATURE (16)..(16): X can be S or I
YIXYSGXTNXNPXLKX

SEQ ID NO: 103 - protein, 14 aa, Artificial Sequence
  HCDR3
  MISC_FEATURE (1)..(1): X can be D or H
XLLYKWNYVEGFDI

SEQ ID NO: 104 - protein, 5 aa, Artificial Sequence
  HCDR1
  MISC_FEATURE (1)..(1): X can be S, N, or R
  MISC_FEATURE (3)..(3): X can be G or D
  MISC_FEATURE (4)..(4): X can be M, T or I
XYXXH

SEQ ID NO: 105 - protein, 17 aa, Artificial Sequence
  HCDR2
  MISC_FEATURE (7)..(7): X can be S or N
  MISC_FEATURE (10)..(10): X can be Y, F, or H
  MISC_FEATURE (12)..(12): X can be A, E, or V
VISYDGXNKXYXDSVKG

SEQ ID NO: 106 - protein, 9 aa, Artificial Sequence
  HCDR3
  MISC_FEATURE (3)..(3): X can be G or D
ERXWDVFDI

SEQ ID NO: 107 - protein, 5 aa, Artificial Sequence
  HCDR1
  MISC_FEATURE (1)..(1): X can be N
  MISC_FEATURE (3)..(3): X can be G or A
XYXMH

SEQ ID NO: 108 - protein, 17 aa, Artificial Sequence
  HCDR2
  MISC_FEATURE (5)..(5): X can be D or H
  MISC_FEATURE (8)..(8): X can be N or D
VISYXGSXKYYADSVKG

SEQ ID NO: 109 - protein, 10 aa, Artificial Sequence
  HCDR3
  MISC_FEATURE (7)..(7): X can be V or A
DGDNWDXFDI

(19) United States
(12) Patent Application Publication
(10) Pub. No.: US 2022/0363761 A1
(43) Pub. Date: Nov. 17, 2022

(54) MULTISPECIFIC BINDING MOIETIES COMPRISING NOVEL PD-1 BINDING DOMAINS

(71) Applicants: Merus N.V., Utrecht (NL); Incyte Corporation, Wilmington, DE (US)

(72) Inventors: Simon Edward PLYTE, Utrecht (NL); Patrick MAYES, Wilmington, DE (US); Horacio G. NASTRI, Wilmington, DE (US); Shaun M. STEWART, Wilmington, DE (US)

(21) Appl. No.: 17/708,901

(22) Filed: Mar. 30, 2022

(30) Foreign Application Priority Data
Mar. 31, 2021 (NL) NL2027893

(51) Int. Cl. C07K 16/28; A61P 35/00

(52) U.S. Cl. CPC C07K 16/2818; A61P 35/00; A61K 2039/505

(57) ABSTRACT

The present disclosure relates to multispecific binding moieties comprising novel PD-1 binding domains that have a higher binding affinity for human PD-1 than a reference PD-1 binding domain. Such multispecific binding moieties further provide a comparable, or equal or higher, potency in blocking ligand binding to human PD-1 than a reference PD-1 antibody. The present disclosure in particular relates to multispecific binding moieties comprising a novel PD-1 binding domain and a LAG-3 binding domain. Also provided is a method for treating a disease, in particular a disease associated with a suppressed immune system, such as cancer, with a multispecific binding moiety of the present disclosure. The present disclosure further relates to a vector and cell comprising nucleic acids encoding a novel PD-1 binding domain and a LAG-3 binding domain.

### FIELD

The present disclosure relates to the field of antibodies. In particular it relates to the field of therapeutic antibodies for the treatment of diseases involving aberrant cells. More in particular it relates to multispecific binding moieties comprising novel binding domains that bind human PD-1.

### BACKGROUND

Cancer is still a major cause of death in the world, in spite of the many advances that have been made in the treatment of the disease and the increased knowledge of the molecular events that lead to cancer. Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases the tumors in the patients stop growing or temporarily shrink (referred to as remission) only to start proliferating again, sometimes more rapidly (referred to as relapse), and become increasingly more difficult to treat. Over the past years, the focus of cancer drug development has moved away from broadly cytotoxic chemotherapy to targeted cytostatic therapies with less toxicity. Treatment of advanced cancer with targeted therapies has been validated clinically in leukemia and some other cancers. However, in a majority of carcinomas, targeted approaches are still proving not effective enough to completely abolish cancer in the majority of the patients.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

A developing class of therapeutic antibodies are bispecific antibodies, which comprise two different binding sites that bind different antigens or different epitopes on the same antigen. Bispecific antibodies can be designed for several applications. Firstly, bispecific antibodies may provide greater tissue-specificity than a monospecific antibody. Several tumor-associated antigens are not only (over)expressed by tumor cells but are also expressed on normal, healthy cells. A bispecific antibody directed against two different tumor-associated antigens involved in a particular type of cancer can specifically target the antibody to the tumor site where the antibody induces tumor cell killing, thereby preventing binding to non-tumor cells expressing only one of the antigens and thus reducing off-site toxicity. Other mechanisms of action include for instance the engagement of immune cells to tumor cells, and the disruption of two signaling pathways required for tumor growth.

Immune checkpoint proteins, like for instance PD-1, PD-L1, CTLA-4, LAG-3, and TIM-3, are an interesting target for antibody therapy. To date, a number of monospecific antibodies targeting PD-1 have been described, as well as certain bispecific antibodies comprising a PD-1 targeting binding domain. However, each of these bispecific antibodies has its own challenges in the production of an effective therapeutic drug. There thus remains a need for the development of novel, effective PD-1xLAG-3 bispecific antibodies.

### SUMMARY

One of the objects of the present disclosure is to provide a new pharmaceutical agent for the treatment of human disease, in particular for the treatment of cancer. This object is met by the provision of multispecific binding moieties comprising novel anti-human PD-1 binding domains, and in particular by bispecific antibodies comprising a novel anti-human PD-1 binding domain and an anti-human LAG-3 binding domain.

In certain embodiments, the present disclosure provides a multispecific binding moiety comprising an anti-human PD-1 binding domain having higher binding affinity for human PD-1 than a reference anti-human PD-1 binding domain, wherein the reference anti-human PD-1 binding domain comprises a heavy chain variable region having an amino acid sequence as set forth in SEQ ID NO: 34 and a light chain variable region having an amino acid sequence as set forth in SEQ ID NO: 35.

In certain embodiments, the present disclosure also provides a multispecific binding moiety comprising an anti-human PD-1 binding domain, wherein the anti-human PD-1 binding domain provides at least comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 34 and two light chain variable regions having an amino acid sequence as set forth in SEQ ID NO: 35.

In certain embodiments, the present disclosure further provides a multispecific antibody comprising a PD-1 binding domain as described herein and a binding domain that binds to human LAG-3.

In certain embodiments, the present disclosure further provides a pharmaceutical composition comprising an effective amount of a multispecific binding moiety as described herein.

In certain embodiments, the present disclosure also provides for the multispecific binding moiety as described herein, and pharmaceutical composition as described herein, for use in the treatment of—a disease, for example a disease associated with a suppressed immune system or cancer.

In certain embodiments, the present disclosure provides a method for treating a disease, comprising administering an effective amount of a multispecific binding moiety, or pharmaceutical composition, as described herein, to an individual in need thereof.

In certain embodiments, the present disclosure provides a method for treating cancer, comprising administering an effective amount of a multispecific binding moiety, or pharmaceutical composition, as described herein, to an individual in need thereof.

In certain embodiments, the present disclosure further provides a vector comprising a nucleic acid sequence encoding the heavy chain variable region of an anti-human PD-1 binding domain as described herein, and a nucleic acid sequence encoding the heavy chain variable region of an anti-human LAG-3 binding domain as described herein.

In certain embodiments, the present disclosure further provides a cell comprising a nucleic acid sequence encoding the heavy chain variable region of an anti-human PD-1 binding domain as described herein, and a nucleic acid sequence encoding the heavy chain variable region of an anti-human LAG-3 binding domain as described herein.

The present disclosure further provides a cell producing a multispecific binding moiety as described herein.

In certain embodiments, the present disclosure provides a method for producing a multispecific binding moiety as described herein, as well as a method for producing variants thereof

### DETAILED DESCRIPTION

The present disclosure describes several anti-human PD-1 binding domains, the heavy chain variable region having an amino acid sequence as set forth in SEQ ID NOS: 1-8, and multispecific binding moieties comprising such anti-human PD-1 binding domains.

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used with a certain level of success to treat some types of cancer.

LAG-3 is known under a number of different names such as Lymphocyte Activating 3; Lymphocyte-Activation Gene 3; CD223 Antigen; Protein FDC; CD223; LAG-3; or FDC. External Ids for LAG3 are: HGNC: 6476; Entrez Gene: 3902; Ensembl: ENSG00000089692; OMIM: 153337; and UniProtKB: P18627. LAG-3 is closely related to CD4. LAG-3 is located on the human chromosome 12 (12p13.32) adjacent to the CD4 gene, and its sequence is approximately 20% identical to CD4. The LAG-3 protein binds a nonholomorphic region of major histocompatibility complex 2 (MHC class II) with greater affinity than CD4. LAG-3 is one of the various immune-checkpoint receptors that are coordinately upregulated on both regulatory T cells (Tregs) and anergic T cells. LAG-3 can negatively regulated T cell proliferation, activation and homeostasis.

In certain embodiments, the anti-human PD-1 binding domain of a multispecific binding moiety comprises at least a heavy chain variable region and a light chain variable region. The light chain variable region can be any suitable light chain variable region as described further herein. In certain embodiments, the light chain variable region preferably is a light chain variable region of a light chain that is capable of pairing with multiple heavy chains having different epitope specificities. Such light chain is also referred to in the art as a “common light chain”.

In certain embodiments, the present disclosure provides a multispecific binding moiety comprising an anti-human PD-1 binding domain, wherein the anti-human PD-1 binding domain has higher binding affinity for human PD-1 than a reference anti-human PD-1 binding domain, wherein the reference anti-human PD-1 binding domain comprises a heavy chain variable region having an amino acid sequence as set forth in SEQ ID NO: 34 and a light chain variable region having an amino acid sequence as set forth in SEQ ID NO: 35.

In certain embodiments, the present disclosure provides a multispecific binding moiety comprising an anti-human PD-1 binding domain, in particular a single anti-human PD-1 binding domain, wherein the multispecific binding moiety has higher binding affinity for human PD-1 than a reference anti-human PD-1 antibody, wherein the reference anti-human PD-1 antibody comprises two heavy chain variable regions having an amino acid sequence as set forth in SEQ ID NO: 34 and two light chain variable regions having an amino acid sequence as set forth in SEQ ID NO: 35.

Determining if an anti-human PD-1 binding domain has a higher binding affinity for human PD-1 than the reference anti-human PD-1 binding domain can be done by measuring the binding affinity of both anti-human PD-1 binding domains in the same type of assay, using the same assay conditions. Thus, in certain embodiments, the binding affinity of the anti-human PD-1 binding domain or of the multispecific binding moiety, and the binding affinity of the reference anti-human PD-1 binding domain or of the reference anti-human PD-1 antibody, are measured in the same type of assay, using the same assay conditions. In certain embodiments, the assay is an assay that uses surface plasmon resonance (SPR) to measure binding affinity, such as the biosensor system of Biacore®, or Solution Equilibrium Titration (SET) (see Friguet B et al. (1985) J. Immunol Methods; 77(2): 305-319, and Hanel C et al. (2005) Anal Biochem; 339(1): 182-184).The binding affinity values of the PD-1 binding domains or of the multispecific binding moieties as provided herein are obtained with the method described in Example 4.

In brief, Example 4 describes performing SPR using a Biacore 8K instrument at 25° C. Anti-human Fc antibodies are immobilized via amine coupling on flow cells of an S series sensor chip CMS with immobilization levels of ˜9000 RU. The desired capturing level (100-150 RU) of anti-PD-1 antibodies is achieved by flowing pre-determined concentration of anti-PD-1 antibodies through the active flow cell of each channel for 60 seconds with 10μL/min flow rate. A PD-1 three-fold serial dilution concentration series (total 7 concentrations, highest at 300 nM) and running buffer is injected for 240 seconds (association time) immediately followed by running buffer for 480 seconds (dissociation time) at a flow rate of 45 μL/min. Surface is regenerated with 30-second injection of 3 M MgCl₂ with 30 μL/min flow rate. Binding kinetics and affinity parameters are obtained from a global fit of the data to 1 to 1 binding model.

Preferably, SPR is performed with the anti-human PD-1 binding domains in an IgG format, measuring the binding affinity of its monovalent interaction with PD-1.

In certain embodiments, the anti-human PD-1 binding domain or multispecific binding moiety has at least a ten-fold higher binding affinity for human PD-1 than the reference anti-human PD-1 binding domain or reference anti-human PD-1 antibody, as measured by SPR as described herein, for instance as described in Example 4. In certain embodiments, the anti-human PD-1 binding domain or multispecific binding moiety has a ten to fifty, ten to forty, ten to thirty, or ten to twenty, fold higher binding affinity for human PD-1 than the reference anti-human PD-1 binding domain or reference anti-human PD-1 antibody, as measured by SPR as described herein, for instance as described in Example 4. In certain embodiments, the anti-human PD-1 binding domain or multispecific binding moiety has a ten-fold higher binding affinity for human PD-1 than the reference anti-human PD-1 binding domain or reference anti-human PD-1 antibody, as measured by SPR as described herein, for instance as described in Example 4.

In certain embodiments, the anti-human PD-1 binding domain or multispecific binding moiety has a binding affinity for human PD-1 in a range of about 0.1-1.0 nM, in particular in a range of about 0.3-0.8 nM, more in particular in a range of about 0.38-0.78 nM, as measured by SPR as described herein, for instance as described in Example 4. In certain embodiments, the anti-human PD-1 binding domain or multispecific binding moiety has a binding affinity for human PD-1 in a range of 0.1-1.0 nM, in particular in a range of 0.3-0.8 nM, more in particular in a range of 0.38-0.78 nM, as measured by SPR as described herein, for instance as described in Example 4. In certain embodiments, the binding affinity is the binding affinity of a monovalent interaction with PD-1.

In certain embodiments, the binding affinity is measured with both the anti-human PD-1 binding domain of the present disclosure and the reference anti-human PD-1 binding domain in a bivalent monospecific IgG format. In certain embodiments, the binding affinity is measured with both the anti-human PD-1 binding domain of the present disclosure and the reference anti-human PD-1 binding domain in a bivalent bispecific IgG format. In certain embodiments, the binding affinity is measured with the anti-human PD-1 binding domain of the present disclosure in a bivalent bispecific IgG format and the reference anti-human PD-1 binding domain in a bivalent monospecific IgG format. A bivalent bispecific IgG format may for instance comprise a PD-1 binding domain of the present disclosure, or a reference anti-human PD-1 binding domain, and a binding domain that binds an unrelated target.

In certain embodiments, the present disclosure also provides a multispecific binding moiety comprising an anti-human PD-1 binding domain, in particular a single anti-human PD-1 binding domain, wherein the anti-human PD-1 binding domain provides at least comparable, or equal or higher, potency in blocking ligand binding to PD-1 than a reference anti-human PD-1 antibody, wherein the reference anti-human PD-1 antibody comprising two heavy chain variable regions having an amino acid sequence as set forth in SEQ ID NO: 34 and two light chain variable regions having an amino acid sequence as set forth in SEQ ID NO: 35.

In certain embodiments, the present disclosure also provides a multispecific binding moiety comprising an anti-human PD-1 binding domain, in particular a single anti-human PD-1 binding domain, wherein the multispecific binding moiety has at least comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 34 and two light chain variable regions having an amino acid sequence as set forth in SEQ ID NO: 35.

Determining if an anti-human PD-1 binding domain or multispecific binding moiety provides a comparable, or equal or higher, potency in blocking ligand binding to PD-1 than the reference anti-human PD-1 antibody can be done by measuring the potency of both the anti-human PD-1 binding domain or of the multispecific binding moiety and the reference antibody in the same type of assay, using the same assay conditions. Thus, in certain embodiments, the potency in blocking ligand binding to PD-1 of the anti-human PD-1 binding domain of the multispecific binding moiety or of the multispecific binding moiety, and the potency in blocking ligand binding to PD-1 of the reference anti-human PD-1 binding antibody, are measured in the same type of assay, using the same assay conditions. In certain embodiments the assay is a PD-1/PD-L1 reporter assay or a PD-1/LAG-3 reporter assay. The potency data of the PD-1 binding domains or of the multispecific binding moieties provided herein is obtained with the PD-1/PD-L1 reporter assay as described in Example 2, and with the PD-1/LAG-3 reporter assay as described in Example 5.

In brief, the PD-1/PD-L1 reporter assay described in Example 2 is performed using PD-L1 aAPC/CHO-K1 cells, which are CHO-K1 cells expressing human PD-L1 and an engineered cell surface protein designed to activate cognate TCRs in an antigen-independent manner, and Jurkat T cells expressing human PD-1 and a luciferase reporter driven by an NFAT response element (NFAT-RE). Assay plates comprising the PD-L1 cells or PBS are incubated overnight at 37° C., 5% CO₂ and 95% Relative Humidity. After incubation, wells are emptied and test and control IgG added in serial dilution, starting with 10 μg/ml and performing 6-step 4-fold titration. A basal control, which is control without IgG is also prepared. IgGs of which activities need to be compared directly are incubated on the same plate. Jurkat T cells are added, and assay plates are incubated for 6 hours at 37° C., 5% CO₂ and 95% Relative Humidity. Following 6 hours of incubation, plates are left at room temperature for 10 min, and luciferase activity is measured.

In brief, the PD-1/LAG-3 reporter assay described in Example 5 is performed using PD-L1 Raji cells and Jurkat PD-1 and LAG-3 effector cells. 25 μl of test and control IgG in 6-fold serial dilution starting between 6-300 μg/ml, with a dilution factor between 2 and 10 (final assay concentration starting between 20-100 μg/ml) is added to assay plates containing 25 μl Jurkat PD-1 and LAG-3 effector cells or PBS. IgGs of which activities need to be compared directly are incubated on the same assay plate. An equal volume of PD-L1 Raji cell suspension was mixed with the same volume of SED solution (100 ng/ml of Staphylococcal enterotoxin D), and 25 μl of Raji/SED mix is added to the assay plates. Assay plates are incubated for 6 hours at 37° C., 5% CO₂ and 95% Relative Humidity. After 6 hours of incubation, assay plates are left at room temperature for 10 minutes, and luciferase activity is measured.

Preferably, the anti-human PD-1 binding domain of the present disclosure and the reference anti-human PD-1 binding domain are used at the same concentration, preferably both in bivalent monospecific IgG format.

In certain embodiments, a comparable potency in PD-1 ligand blocking activity is a potency within a 5 fold range of the potency in blocking ligand binding to PD-1 of the reference anti-human PD-1 antibody, and includes a 5, 4, 3 and 2 fold, preferably a 3 fold, deviation, from the potency in blocking ligand binding to PD-1 of the reference anti-human PD-1 antibody.

In certain embodiments, a higher potency in PD-1—ligand blocking activity is a potency that is a 5, 4, 3, or 2 fold, preferably a 3 fold, higher potency than the potency in blocking ligand binding to PD-1 of the reference anti-human PD-1 antibody. In certain embodiments, the at least comparable, or equal or higher potency in PD-1—ligand blocking activity is a potency that is a 1.1-2.0 fold, preferably a 1.2-1.8 or 1.2-1.6 fold, more preferably a 1.2-1.4 fold, higher potency than the potency in blocking ligand binding to PD-1 of the reference anti-human PD-1 antibody.

The reference anti-human PD-1 binding domain is the PD-1 binding domain of a nivolumab analog antibody, preferably produced using the same production method as the anti-human PD-1 binding domain of the multispecific binding moiety subject to comparison. The reference anti-human PD-1 binding antibody is a nivolumab analog antibody, preferably produced using the same production method as the multispecific binding moiety subject to comparison. A nivolumab analog antibody has the same heavy chain variable region sequence (SEQ ID NO: 20) as nivolumab. A nivolumab analog antibody has the same light chain variable region sequence (SEQ ID NO: 21) as nivolumab.

In certain embodiments, the anti-human PD-1 binding domain of the multispecific binding moiety comprises a heavy chain variable region, wherein the heavy chain variable region comprises the heavy chain CDR1 (HCDR1), heavy chain CDR2 (HCDR2), and heavy chain CDR3 (HCDR3) of one of the heavy chain variable regions having an amino acid sequence as set forth in SEQ ID NOS: 1-8.

CDR sequences can be defined using different methods, including, but not limited to, according to the Kabat numbering scheme (Kabat et al., J. Biol. Chem.252:6609-6616 (1977); and/or Kabat et al., U.S. Dept. of Health and Human Services, “Sequences of proteins of immunological interest” (1991)), the Chothia numbering scheme (Chothia et al., J. Mol. Bio1.196:901-917 (1987); Chothia et al., Nature 342: 877-883, 1989; and/or Al-Lazikani B. et al., J. Mol. Biol., 273: 927-948 (1997)), the numbering system of Honegger and Plukthun (Honegger and Plückthun, J. Mol. Biol., 309:657-670 (2001)), the numbering system of MacCallum (MacCallum et al., J. Mol. Biol. 262:732-745 (1996); and/or Abhinandan and Martin, Mol. Immunol., 45: 3832-3839 (2008)), the numbering system of Lefranc (Lefranc M. P. et al., Dev. Comp. Immunol., 27: 55-77 (2003); and/or Honegger and Plückthun, J. Mol. Biol., 309:657-670 (2001)), or according to IMGT (discussed in Giudicelli et al., Nucleic Acids Res. 25: 206-21 1 (1997)).

Each of these numbering schemes base their definition of CDRs on a predicted contribution of amino acid residues in the heavy or light chain variable region to antigen binding. Hence, each method to identify CDRs can be used to identify the CDRs of the binding domains of the present disclosure. In certain embodiments, the heavy chain CDRs of a binding domain of the present disclosure is according to Kabat, Chothia, or IMGT. In certain embodiments, the heavy chain CDRs of a binding domain of the present disclosure is according to Kabat. In certain embodiments, the heavy chain CDRs of a binding domain of the present disclosure is according to Chothia. In certain embodiments, the heavy chain CDRs of a binding domain of the present disclosure is according to IMGT.

In certain embodiments, the anti-human PD-1 binding domain comprises a heavy chain variable region, wherein the heavy chain variable region comprises a heavy chain CDR1 (HCDR1) from a heavy chain variable region having an amino acid sequence from the group consisting of SEQ ID NOS:1-8; a heavy chain CDR2 (HCDR2) from a heavy chain variable region having an amino acid sequence from the group consisting of SEQ ID NOS:1-8; and a heavy chain CDR3 (HCDR3) from a heavy chain variable region having an amino acid sequence from the group consisting of SEQ ID NOS: 1-8.

The HCDRs according to Kabat are indicated in bold and underlined in the list of sequences provided herein.

In certain embodiments, the heavy chain variable region of the anti-human PD-1 binding domain of the multispecific binding moiety comprises:
heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
heavy chain CDR1 (HCDR1), heavy chain CDR2 (HCDR2), and heavy chain CDR3 (HCDR3), having an amino acid sequence as set forth in SEQ ID NO: 39, SEQ ID NO: 40, and SEQ ID NO: 41, respectively;
heavy chain CDR1 (HCDR1), heavy chain CDR2 (HCDR2), and heavy chain CDR3 (HCDR3), having an amino acid sequence as set forth in SEQ ID NO: 42, SEQ ID NO: 43, and SEQ ID NO: 44, respectively;
heavy chain CDR1 (CDR1), heavy chain CDR2 (HCDR2) and heavy chain CDR3 (HCDR3), having an amino acid sequence as set forth in SEQ ID NO: 45, SEQ ID NO: 46, and SEQ ID NO: 47, respectively;
heavy chain CDR1 (HCDR1), heavy chain CDR2 (HCDR2), and heavy chain CDR3 (HCDR3), having an amino acid sequence as set forth in SEQ ID NO: 48, SEQ ID NO: 49, and SEQ ID NO: 50, respectively;
heavy chain CDR1 (HCDR1), heavy chain CDR2 (HCDR2), and heavy chain CDR3 (HCDR3), having an amino acid sequence as set forth in SEQ ID NO: 51, SEQ ID NO: 52, and SEQ ID NO: 53, respectively;
heavy chain CDR1 (HCDR1), heavy chain CDR2 (HCDR2), and heavy chain CDR3 (HCDR3), having an amino acid sequence as set forth in SEQ ID NO: 54, SEQ ID NO: 55, and SEQ ID NO: 56, respectively; or
heavy chain CDR1 (HCDR1), heavy chain CDR2 (HCDR2), and heavy chain CDR3 (HCDR3), having an amino acid sequence as set forth in SEQ ID NO: 57, SEQ ID NO: 58, and SEQ ID NO: 59, respectively;
wherein each of the HCDRs may comprise at most three, two, or one amino acid substitutions.

In certain embodiments, the heavy chain variable region of the anti-human PD-1 binding domain of the multispecific binding moiety comprises:
heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
heavy chain CDR1 (HCDR1), heavy chain CDR2 (HCDR2), and heavy chain CDR3 (HCDR3), having an amino acid sequence as set forth in SEQ ID NO: 39, SEQ ID NO: 40, and SEQ ID NO: 41, respectively;
heavy chain CDR1 (HCDR1), heavy chain CDR2 (HCDR2), and heavy chain CDR3 (HCDR3), having an amino acid sequence as set forth in SEQ ID NO: 42, SEQ ID NO: 43, and SEQ ID NO: 44, respectively;
heavy chain CDR1 (HCDR1), heavy chain CDR2 (HCDR2), and heavy chain CDR3 (HCDR3), having an amino acid sequence as set forth in SEQ ID NO: 45, SEQ ID NO: 46, and SEQ ID NO: 47, respectively;
heavy chain CDR1 (HCDR1), heavy chain CDR2 (HCDR2), and heavy chain CDR3, (HCDR3), having an amino acid sequence as set forth in SEQ ID NO: 48, SEQ ID NO: 49, and SEQ ID NO: 50, respectively;
heavy chain CDR1 (HCDR1), heavy chain CDR2 (HCDR2), and heavy chain CDR3 (HCDR3), having an amino acid sequence as set forth in SEQ ID NO: 51, SEQ ID NO: 52, and SEQ ID NO: 53, respectively;
heavy chain CDR1 (HCDR1), heavy chain CDR2 (HCDR2), and heavy chain CDR3 (HCDR3), having an amino acid sequence as set forth in SEQ ID NO: 54, SEQ ID NO: 55, and SEQ ID NO: 56, respectively; or
heavy chain CDR1 (HCDR1), heavy chain CDR2 (HCDR2), and heavy chain CDR3 (HCDR3), having an amino acid sequence as set forth in SEQ ID NO: 57, SEQ ID NO: 58, and SEQ ID NO: 59, respectively.

In certain embodiments, a PD-1 binding domain of a multispecific binding moiety of the present disclosure also includes PD-1 binding domain variants, wherein each of the HCDRs may comprise at most three, two, or one amino acid substitutions. In certain embodiments, only one or two HCDRs may comprise at most three, two, or one amino acid substitutions.

For example, suitable positions for introducing an amino acid variation include, but are not limited to, the first, second, and/or fourth amino acid of HCDR1; the third, seventh, eighth, ninth, tenth, eleventh, thirteenth, fourteenth, and/or sixteenth amino acid of HCDR2; and/or the sixth and/or thirteenth amino acid of HCDR3. CDR sequences according to Kabat are indicated in bold and underlined in the list of sequences provided herein.

In certain embodiments, the present disclosure thus also provides an anti-human PD-1 binding domain comprising:
HCDR1 having amino acid sequence X₁X₂FX₃S, wherein
   X₁ can be F, Y, T, or H;
   X₂ can be Y, Q, E, H, or D;
   X₃ can be W, or Y; and/or
HCDR2 having amino acid sequence YIX₁YSGX₂X₃X₄X₅X₆PX₇X₈KX₉, wherein
   X₁ can be Y, V, or I;
   X₂ can be S, or G;
   X₃ can be T, Y, S, H, N, W, L, or Q;
   X₄ can be S, or N;
   X₅ can be F, V, or L;
   X₆ can be N, or S;
   X₇ can be S or A;
   X₈ can be F or L;
   X₉ can be S, T, G, D, R, or N; and/or
HCDR3 having amino acid sequence GGYTGX₁GGDWFDX₂, wherein
   X₁ can be Y, H, V, or A;
   X₂ can be P, V, Y, W, F, T, Q, H, or S.

Other suitable positions for introducing an amino acid variation include, but are not limited to, the second, third, fourth, and/or fifth amino acid of HCDR1; the third, fourth, fifth, sixth, eighth, ninth, tenth, eleventh, twelfth, thirteenth, fourteenth, fifteenth, sixteenth and/or seventeenth amino acid of HCDR2; and/or the first, second, sixth, seventh, ninth, tenth, fourteenth, fifteenth, sixteenth and/or eighteenth amino acid of HCDR3. CDR sequences according to Kabat are indicated in bold and underlined in the list of sequences provided herein.

In certain embodiments, the present disclosure thus also provides an anti-human PD-1 binding domain comprising:
HCDR1 having amino acid sequence RX₁X₂X₃X₄, wherein
   X₁ can be F, or Y;
   X₂ can be T, A, or V;
   X₃ can be M, L, or V;
   X₄ can be S, H, N, V, or T; and/or
HCDR2 having amino acid sequence
WIX₁X₂X₃X₄GX₅X₆X₇X₈X₉X₁₀X₁₁X₁₂X₁₃X₁₄, wherein
   X₁ can be N, or D;
   X₂ can be P, S, or T;
   X₃ can be N, or Q;
   X₄ can be T, or D;
   X₅ can be N, S, T, K, L, or E;
   X₆ can be P, Y, A, H, or F;
   X₇ can be T, or S;
   X₈ can be Y, F, or H;
   X₉ can be A, G, V, or F;
   X₁₀ can be Q, R, N, L, T, or S;
   X₁₁ can be D, A, G, or S;
   X₁₂ can be F, V, or A;
   X₁₃ can be T, K, H, G;
   X₁₄ can be G, N, E, or D; and/or
HCDR3 having amino acid sequence X₁X₂GYCX₃X₄DX₅CYPNX₆X₇X₈DX₉, wherein
   X₁ can be I, S, or V;
   X₂ can be L, Q, or N;
   X₃ can be N, G, S, or D;
   X₄ can be T, S, P, N, or E;
   X₅ can be N, or I;
   X₆ can be W, G, Q, H, W, A, or L;
   X₇ can be I, V, or L;
   X₈ can be F, L, or I;
   X₉ can be Y, S, N, I, R, H, V, T, K, A, or L.

In certain embodiments, the anti-human PD-1 binding domain of the multispecific binding moiety of the present disclosure comprises a heavy chain variable region having an amino acid sequence as set forth in any one of SEQ ID NOS: 1-8, or having at least 80%, preferably 85%, more preferably 90%, or most preferably 95% sequence identity thereto.

In certain embodiments, a PD-1 binding domain of the multispecific binding moiety of the present disclosure also includes PD-1 binding domain variants, which, in addition to the variations in the HCDRs referred to above, comprise one or more variations in the framework regions. In certain embodiments, a PD-1 binding domain variant of the multispecific binding moiety of the present disclosure comprises no variations in the CDR regions but comprises one or more variations in the framework regions. Such variants have at least 80%, preferably 85%, more preferably 90%, or most preferably 95% sequence identity to the sequences disclosed herein, and are expected to retain PD-1 binding specificity. Thus, in certain embodiments, a PD-1 binding domain of the multispecific binding moiety of the present disclosure comprises:
a heavy chain variable region having at least 80%, preferably 85%, more preferably 90%, or most preferably 95% sequence identity to the amino acid sequence as set forth in SEQ ID NO: 1, which heavy chain variable region comprises a HCDR1 amino acid sequence as set forth in SEQ ID NO: 36; a HCDR2 amino acid sequence as set forth in SEQ ID NO: 37; and a HCDR3 amino acid sequence as set forth in SEQ ID NO: 38;
a heavy chain variable region having at least 80%, preferably 85%, more preferably 90%, or most preferably 95% sequence identity to the amino acid sequence as set forth in SEQ ID NO: 2, which heavy chain variable region comprises a HCDR1 amino acid sequence as set forth in SEQ ID NO: 39; a HCDR2 amino acid sequence as set forth in SEQ ID NO: 40; and a HCDR3 amino acid sequence as set forth in SEQ ID NO: 41;
a heavy chain variable region having at least 80%, preferably 85%, more preferably 90%, or most preferably 95% sequence identity to the amino acid sequence as set forth in SEQ ID NO: 3, which heavy chain variable region comprises a HCDR1 amino acid sequence as set forth in SEQ ID NO: 42; a HCDR2 amino acid sequence as set forth in SEQ ID NO: 43; and a HCDR3 amino acid sequence as set forth in SEQ ID NO: 44;
a heavy chain variable region having at least 80%, preferably 85%, more preferably 90%, or most preferably 95% sequence identity to the amino acid sequence as set forth in SEQ ID NO: 4, which heavy chain variable region comprises a HCDR1 amino acid sequence as set forth in SEQ ID NO: 45; a HCDR2 amino acid sequence as set forth in SEQ ID NO: 46; and a HCDR3 amino acid sequence as set forth in SEQ ID NO: 47;
a heavy chain variable region having at least 80%, preferably 85%, more preferably 90%, or most preferably 95% sequence identity to the amino acid sequence as set forth in SEQ ID NO: 5, which heavy chain variable region comprises a HCDR1 amino acid sequence as set forth in SEQ ID NO: 48; a HCDR2 amino acid sequence as set forth in SEQ ID NO: 49; and a HCDR3 amino acid sequence as set forth in SEQ ID NO: 50;
a heavy chain variable region having at least 80%, preferably 85%, more preferably 90%, or most preferably 95% sequence identity to the amino acid sequence as set forth in SEQ ID NO: 6, which heavy chain variable region comprises a HCDR1 amino acid sequence as set forth in SEQ ID NO: 51; a HCDR2 amino acid sequence as set forth in SEQ ID NO: 52; and a HCDR3 amino acid sequence as set forth in SEQ ID NO: 53;
a heavy chain variable region having at least 80%, preferably 85%, more preferably 90%, or most preferably 95% sequence identity to the amino acid sequence as set forth in SEQ ID NO: 7, which heavy chain variable region comprises a HCDR1 amino acid sequence as set forth in SEQ ID NO: 54; a HCDR2 amino acid sequence as set forth in SEQ ID NO: 55; and a HCDR3 amino acid sequence as set forth in SEQ ID NO: 56; or
a heavy chain variable region having at least 80%, preferably 85%, more preferably 90%, or most preferably 95% sequence identity to the amino acid sequence as set forth in SEQ ID NO: 8, which heavy chain variable region comprises a HCDR1 amino acid sequence as set forth in SEQ ID NO: 57; a HCDR2 amino acid sequence as set forth in SEQ ID NO: 58; and a HCDR3 amino acid sequence as set forth in SEQ ID NO: 59.

In certain embodiments, a PD-1 binding domain of the multispecific binding moiety of the present disclosure comprises a light chain variable region. An example of a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 60, SEQ ID NO: 61, and SEQ ID NO: 62, respectively, wherein each of the LCDRs may comprise at most three, two, or one amino acid substitutions. In certain embodiments, a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 60, SEQ ID NO: 61, and SEQ ID NO: 62, respectively. In certain embodiments, such light chain variable region may comprise a light chain variable region having an amino acid sequence as set forth in SEQ ID NO:24, or having at least 80%, preferably 85%, more preferably 90%, or most preferably 95% sequence identity thereto. A light chain or light chain variable region comprising these LCDRs and/or light chain variable region is the light chain referred to in the art as VK1-39/JK1. This is a common light chain.

In certain embodiments, a PD-1 binding domain of the multispecific binding moiety of the present disclosure comprises a light chain variable region having at least 80%, preferably 85%, more preferably 90%, or most preferably 95% sequence identity to the amino acid sequence as set forth in SEQ ID NO: 24, which light chain variable region comprises a LCDR1 amino acid sequence as set forth in SEQ ID NO: 60; a LCDR2 amino acid sequence as set forth in SEQ ID NO: 61; and a LCDR3 amino acid sequence as set forth in SEQ ID NO: 62.

The term ‘common light chain’ according to the present disclosure refers to a light chain that is capable of pairing with multiple different heavy chains, i.e. heavy chains having different antigen or epitope binding specificities. A common light chain is particularly useful in the generation of, for instance, bispecific antibodies, where antibody production is more efficient when both binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 63. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 63, wherein each of the LCDRs may comprise at most three, two, or one amino acid substitutions. In certain embodiments, the light chain comprises a light chain variable region having an amino acid sequence as set forth in SEQ ID NO: 63, or having at least 80%, preferably 85%, more preferably 90%, or most preferably 95% sequence identity thereto; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 64. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 64, wherein each of the LCDRs may comprise at most three, two, or one amino acid substitutions. In certain embodiments, the light chain comprises a light chain variable region having an amino acid sequence as set forth in SEQ ID NO: 64, or having at least 80%, preferably 85%, more preferably 90%, or most preferably 95% sequence identity thereto; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 65. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 65, wherein each of the LCDRs may comprise at most three, two, or one amino acid substitution. In certain embodiments, the light chain comprises a light chain variable region having an amino acid sequence as set forth in SEQ ID NO: 65, or having at least 80%, preferably 85%, more preferably 90%, or most preferably 95% sequence identity thereto; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 66. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 66, wherein each of the LCDRs may comprise at most three, two, or one amino acid substitutions. In certain embodiments, the light chain comprises a light chain variable region having an amino acid sequence as set forth in SEQ ID NO: 66, or having at least 80%, preferably 85%, more preferably 90%, or most preferably 95% sequence identity thereto.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. A preferred amino acid sequence for VK1-39 is given as SEQ ID NO: 67. This is the sequence of the V-region. The V-region can be combined with one of five J-regions. Two preferred joined sequences are indicated as VK1-39/JK1 and VK1-39/JKS; alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. A preferred amino acid sequence for VK3-15 is given as SEQ ID NO: 68. This is the sequence of the V-region. The V-region can be combined with one of five J-regions. A preferred joined sequence is indicated as VK3-15/JK1; alternative name is Vκ3-15*01/IGKκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External

Ids for the gene are HGNC: 5817; Entrez Gene: 28912; Ensembl: ENSG00000239951. A preferred amino acid sequence for VK3-20 is given as SEQ ID NO: 69. This is the sequence of the V-region. The V-region can be combined with one of five J-regions. A preferred joined sequence is indicated as VK3-20/JK1; alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. A preferred amino acid sequence for VL3-21 is given as SEQ ID NO: 70. This is the sequence of the V-region. The V-region can be combined with one of five J-regions. A preferred joined sequence is indicated as VL3-21/JL3; alternative name is IgVλ3-21/IGJκ3 (nomenclature according to the IMGT database worldwide web at imgt.org). This name is exemplary and encompasses allelic variants of the gene segments.

Further, any light chain variable region of a PD-1 antibody available in the art may be used, or any other light chain variable region that can readily be obtained, such as from, for instance, an antibody display library by showing antigen binding activity when paired with a PD-1 binding domain of the invention.

In certain embodiments, a PD-1 binding domain of the multispecific binding moiety of the present disclosure may further comprise a CH1 and CL region. Any CH1 domain may be used, in particular a human CH1 domain. An example of a suitable CH1 domain is provided by the amino acid sequence provided as SEQ ID NO: 29. Any CL domain may be used, in particular a human CL. An example of a suitable CL domain is provided by the amino acid sequence provided as SEQ ID NO: 71.

A “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like V_{HH} and V_{H}), minibodies, scFab, scFv-zipper, nanobodies, DART molecules, TandAb, Fab-scFv, F(ab)′2, F(ab)′2-scFv2, and intrabodies.

In one embodiment, the multispecific binding moiety is a multispecific antibody. A multispecific antibody according to the present disclosure is an antibody, in any antibody format, that comprises at least two binding domains which have specificity for at least two different targets or epitopes. In certain embodiments, a multispecific antibody of the invention is a bispecific antibody. In certain embodiments, a multispecific antibody of the present disclosure may further comprise an Fc region or a part thereof. In certain embodiments, a multispecific binding moiety of the present disclosure is an IgG1 antibody.

In certain embodiments, a multispecific binding moiety of the present disclosure further comprises a binding domain that binds to a cell surface moiety expressed on an immune effector cell.

In certain embodiments, the multispecific binding moiety of the present disclosure comprises a PD-1 binding domain as described herein and an anti-human LAG-3 binding domain. Suitable anti-human LAG-3 binding domains comprise a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 11;
heavy chain CDR1 (HCDR1), heavy chain CDR2 (HCDR2), and heavy chain CDR3 (HCDR3), of a heavy chain variable region having an amino acid sequence as set forth in SEQ ID NO: 12;
heavy chain CDR1 (HCDR1), heavy chain CDR2 (HCDR2), and heavy chain CDR3 (HCDR3), of a heavy chain variable region having an amino acid sequence as set forth in SEQ ID NO: 13,
heavy chain CDR1 (HCDR1), heavy chain CDR2 (HCDR2), and heavy chain CDR3 (HCDR3), of a heavy chain variable region having an amino acid sequence as set forth in SEQ ID NO: 14,
heavy chain CDR1 (HCDR1), heavy chain CDR2 (HCDR2), and heavy chain CDR3 (HCDR3), of a heavy chain variable region having an amino acid sequence as set forth in SEQ ID NO: 15,
heavy chain CDR1 (HCDR1), heavy chain CDR2 (HCDR2), and heavy chain CDR3 (HCDR3), of a heavy chain variable region having an amino acid sequence as set forth in SEQ ID NO: 16, and
heavy chain CDR1 (HCDR1), heavy chain CDR2 (HCDR2), and heavy chain CDR3 (HCDR3), of a heavy chain variable region having an amino acid sequence as set forth in SEQ ID NO: 17,

wherein each of the HCDRs may comprise at most three, two, or one amino acid substitutions. HCDRs are indicated in bold and underlined in the referenced SEQ ID NOs in the listing of sequences provided herein.

In certain embodiments, the anti-human LAG-3 binding domain comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 11;
heavy chain CDR1 (HCDR1), heavy chain CDR2 (HCDR2), and heavy chain CDR3 (HCDR3), of a heavy chain variable region having an amino acid sequence as set forth in SEQ ID NO: 12;
heavy chain CDR1 (HCDR1), heavy chain CDR2 (HCDR2), and heavy chain CDR3 (HCDR3), of a heavy chain variable region having an amino acid sequence as set forth in SEQ ID NO: 13,
heavy chain CDR1 (HCDR1), heavy chain CDR2 (HCDR2), and heavy chain CDR3 (HCDR3), of a heavy chain variable region having an amino acid sequence as set forth in SEQ ID NO: 14,
heavy chain CDR1 (HCDR1), heavy chain CDR2 (HCDR2), and heavy chain CDR3 (HCDR3), of a heavy chain variable region having an amino acid sequence as set forth in SEQ ID NO: 15,
heavy chain CDR1 (HCDR1), heavy chain CDR2 (HCDR2), and heavy chain CDR3 (HCDR3), of a heavy chain variable region having an amino acid sequence as set forth in SEQ ID NO: 16, and
heavy chain CDR1 (HCRD1), heavy chain CRD2 (HCRD2), and a heavy chain CDR3 (HCDR3), of a heavy chain variable region having an amino acid sequence as set forth in SEQ ID NO: 17.

In certain embodiments, a LAG-3 binding domain of the present disclosure includes LAG-3 binding domain variants, wherein each of the HCDRs may comprise at most three, two, or one amino acid substitutions. Such variants are expected to retain LAG-3 binding specificity.

For example, suitable positions for introducing an amino acid variation include, but are not limited to, the second, and/or third amino acid of HCDR1; the third, seventh, tenth, thirteenth, and/or sixteenth amino acid of HCDR2; and/or the first amino acid of HCDR3.

HCDR sequences according to Kabat are indicated in bold and underlined in the list of sequences provided herein.

In certain embodiments, the anti-human LAG-3 binding domain comprises:
HCDR1 having amino acid sequence SX₁X₂WS, wherein
   X₁ can be Y or F;
   X₂ can be Y or S; and/or
HCDR2 having amino acid sequence YIX₁YSGX₂TNX₃NPX₄LKX₅, wherein
   X₁ can be Y or D;
   X₂ can be S, or T;
   X₃ can be Y or F;
   X₄ can be S, or F;
   X₅ can be S or I; and/or
HCDR3 having amino acid sequence X₁LLYKWNYVEGFDI, wherein
   X₁ can be D or H.

For example, suitable positions for introducing an amino acid variation include, but are not limited to, the first, third and/or fourth amino acid of HCDR1; the seventh, tenth, and/or twelfth amino acid of HCDR2; and/or the third amino acid of HCDR3. HCDR sequences according to Kabat are indicated in bold and underlined in the list of sequences provided herein. In certain embodiments, the anti-human LAG-3 binding domain comprises:
HCDR1 having amino acid sequence X₁YX₂X₃H, wherein
   X₁ can be S, N, or R;
   X₂ can be G or D;
   X₃ can be M, T or I; and/or
HCDR2 having amino acid sequence VISYDGX₁NKX₂YX₃DSVKG, wherein
   X₁ can be S or N;
   X₂ can be Y, F, or H;
   X₃ can be A, E, or V; and/or
HCDR3 having amino acid sequence ERX₁WDVFDI, wherein
   X₁ can be G or D.

For example, suitable positions for introducing an amino acid variation include, but are not limited to, the first, and/or third amino acid of HCDR1; the fifth, and/or eighth amino acid of HCDR2; and/or the third amino acid of HCDR3. HCDR sequences according to Kabat are indicated in bold and underlined in the list of sequences provided herein.

In certain embodiments, the anti-human LAG-3 binding domain comprises:
HCDR1 having amino acid sequence X₁YX₂MH, wherein
   X₁ can be S or N;
   X₂ can be G or A; and/or
HCDR2 having amino acid sequence VISYX₁GSX₂KYYADSVKG, wherein
   X₁ can be D or H;
   X₂ can be N or D; and/or
HCDR3 having amino acid sequence DGDNWDX1FDI, wherein

### X₁ can be V or A.

In certain embodiments, an anti-human LAG-3 binding domain of the present disclosure comprises a heavy chain variable region having an amino acid sequence as set forth in any one of SEQ ID NO: 11-17, or having at least 80%, preferably 85%, more preferably 90%, or most preferably 95% sequence identity thereto.

In certain embodiments, a LAG-3 binding domain of the present disclosure also includes LAG-3 binding domain variants, which, in addition to variations in the HCDRs, comprise one or more variations in the framework regions. In certain embodiments, a LAG-3 binding domain variant of the present disclosure comprises no variations in the CDR regions but comprises one or more variations in the framework regions. Such variants have at least 80%, preferably 85%, more preferably 90%, or most preferably 95% sequence identity to the sequences disclosed herein, and are expected to retain LAG-3 binding specificity. Thus, in certain embodiments, a LAG-3 binding domain of the multispecific binding moiety of the present disclosure comprises:
a heavy chain variable region having at least 80%, preferably 85%, more preferably 90%, or most preferably 95% sequence identity to the amino acid sequence as set forth in SEQ ID NO: 11, which heavy chain variable region comprises the HCDR1 amino acid sequence as set forth in SEQ ID NO: 74, HCDR2 amino acid sequence as set forth in SEQ ID NO: 75, and HCDR3 amino acid sequence of the amino acid sequence as set forth in SEQ ID NO: 76;
a heavy chain variable region having at least 80%, preferably 85%, more preferably 90%, or most preferably 95% sequence identity to the amino acid sequence as set forth in SEQ ID NO: 12, which heavy chain variable region comprises the HCDR1 amino acid sequence as set forth in SEQ ID NO: 77, HCDR2 amino acid sequence as set forth in SEQ ID NO: 78, and HCDR3 amino acid sequence of the amino acid sequence as set forth in SEQ ID NO: 79;
a heavy chain variable region having at least 80%, preferably 85%, more preferably 90%, or most preferably 95% sequence identity to the amino acid sequence as set forth in SEQ ID NO: 13, which heavy chain variable region comprises the HCDR1 amino acid sequence as set forth in SEQ ID NO: 80, HCDR2 amino acid sequence as set forth in SEQ ID NO: 81, and HCDR3 amino acid sequence of the amino acid sequence as set forth in SEQ ID NO: 82;
a heavy chain variable region having at least 80%, preferably 85%, more preferably 90%, or most preferably 95% sequence identity to the amino acid sequence as set forth in SEQ ID NO: 14, which heavy chain variable region comprises the HCDR1 amino acid sequence as set forth in SEQ ID NO: 83, HCDR2 amino acid sequence as set forth in SEQ ID NO: 84, and HCDR3 amino acid sequence of the amino acid sequence as set forth in SEQ ID NO: 85;
a heavy chain variable region having at least 80%, preferably 85%, more preferably 90%, or most preferably 95% sequence identity to the amino acid sequence as set forth in SEQ ID NO: 15, which heavy chain variable region comprises the HCDR1 amino acid sequence as set forth in SEQ ID NO: 86, HCDR2 amino acid sequence as set forth in SEQ ID NO: 87, and HCDR3 amino acid sequence of the amino acid sequence as set forth in SEQ ID NO: 88;
a heavy chain variable region having at least 80%, preferably 85%, more preferably 90%, or most preferably 95% sequence identity to the amino acid sequence as set forth in SEQ ID NO: 16, which heavy chain variable region comprises the HCDR1 amino acid sequence as set forth in SEQ ID NO: 89, HCDR2 amino acid sequence as set forth in SEQ ID NO: 90, and HCDR3 amino acid sequence of the amino acid sequence as set forth in SEQ ID NO: 91; or
a heavy chain variable region having at least 80%, preferably 85%, more preferably 90%, or most preferably 95% sequence identity to the amino acid sequence as set forth in SEQ ID NO: 17, which heavy chain variable region comprises the HCDR1 amino acid sequence as set forth in SEQ ID NO: 92, HCDR2 amino acid sequence as set forth in SEQ ID NO: 93, and HCDR3 amino acid sequence of the amino acid sequence as set forth in SEQ ID NO: 94.

In certain embodiments, a LAG-3 binding domain of the present disclosure further comprises a light chain variable region. An example of a suitable light chain variable region is a light chain variable region as described herein, for instance, a light chain variable region as described herein for the PD-1 binding domain of the present disclosure. Light chain variable regions of LAG-3 antibodies available in the art may be used, or any other light chain variable region that can readily be obtained, such as from, for instance, an antibody display library by showing antigen binding activity when paired with a LAG-3 binding domain of the present disclosure. Preferably, a LAG-3 binding domain of the present disclosure comprises a VK1-39/JK1, VK1-39/JKS, VK3-15/JK1, VK3-20/JK1, or VL3-21/JL3 light chain variable region.

In certain embodiments, an anti-human LAG-3 binding domain of the present disclosure may further comprise a CH1 and CL region. Any CH1 domain may be used, in particular a human CH1 domain. An example of a suitable CH1 domain is provided by the amino acid sequence provided as SEQ ID NO: 29. Any CL domain may be used, in particular a human CL. An example of a suitable CL domain is provided by the amino acid sequence provided as SEQ ID NO: 71.

In certain embodiments, a PD-1 binding domain disclosed herein can be combined with any LAG-3 binding domain disclosed herein to produce a multispecific binding moiety of the present disclosure. The present disclosure thus also provides multispecific binding moieties PB1-PB35, as presented in Table 1.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 1; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 15.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 12.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 13.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 14.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 15.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 14.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 7; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 8; and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17.

TABLE 1
Binding moieties comprising combinations of heavy
chain variable regions specific for PD-1 and heavy chain
variable regions specific for LAG-3. Each of PB1-PB35
can be combined with the light chain disclosed herein.

| | SEQ | SEQ | SEQ | SEQ | SEQ | SEQ | SEQ |
|---|---|---|---|---|---|---|---|
| | ID | ID | ID | ID | ID | ID | ID |
| | NO: 11 | NO: 12 | NO: 13 | NO: 14 | NO: 15 | NO: 16 | NO: 17 |
| SEQ | PB1 | PB2 | PB3 | PB4 | PB5 | PB6 | PB19 |
| ID | | | | | | | |
| NO: 1 | | | | | | | |
| SEQ | PB7 | PB8 | PB9 | PB10 | PB11 | PB12 | PB20 |
| ID | | | | | | | |
| NO: 6 | | | | | | | |
| SEQ | PB13 | PB14 | PB15 | PB16 | PB17 | PB18 | PB21 |
| ID | | | | | | | |
| NO: 5 | | | | | | | |
| SEQ | PB22 | PB23 | PB24 | PB25 | PB26 | PB27 | PB28 |
| ID | | | | | | | |
| NO: 7 | | | | | | | |
| SEQ | PB29 | PB30 | PB31 | PB32 | PB33 | PB34 | PB35 |
| ID | | | | | | | |
| NO: 8 | | | | | | | |

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 1, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 15, wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 12, wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 13,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 14,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1

(HCDR1), heavy chain CDR2 (HCDR2), and heavy chain CDR3 (HCDR3), of a heavy chain variable region having an amino acid sequence as set forth in SEQ ID NO: 6, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 15,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5, and
a LAG-3 binding domain of the present disclosure comprising heavy chain CDR1

(HCDR1), heavy chain CDR2 (HCDR2), and heavy chain CDR3 (HCDR3), of a heavy chain variable region having an amino acid sequence as set forth in SEQ ID NO: 14,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 7, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 8, and
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17,
wherein the PD-1 binding domain and LAG-3 binding domain comprise light chain CDR1 (LCDR1) having an amino acid sequence as set forth in SEQ ID NO: 60, light chain CDR2 (LCDR2) having an amino acid sequence as set forth in SEQ ID NO: 61, and light chain CDR3 (LCDR3) having an amino acid sequence as set forth in SEQ ID NO: 62.

In one embodiment, the multispecific binding moiety of the present disclosure comprises:
a PD-1 binding domain of the present disclosure comprising a heavy chain variable region having an amino acid sequence as set forth in SEQ ID NO: 1, and
a LAG-3 binding domain of the present disclosure comprising a heavy chain variable region having an amino acid sequence as set forth in SEQ ID NO: 15.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 12.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 13.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 14.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 15.

In one embodiment, the multispecific binding moiety of the present disclosure comprises:
a PD-1 binding domain of the present disclosure comprising a heavy chain variable region having an amino acid sequence as set forth in SEQ ID NO: 5, and
a LAG-3 binding domain of the present disclosure comprising a heavy chain variable region having an amino acid sequence as set forth in SEQ ID NO: 14.

In one embodiment, the multispecific binding moiety of the present disclosure comprises:
a PD-1 binding domain of the present disclosure comprising a heavy chain variable region having an amino acid sequence as set forth in SEQ ID NO: 7, and
a LAG-3 binding domain of the present disclosure comprising a heavy chain variable region having an amino acid sequence as set forth in SEQ ID NO: 17.

In one embodiment, the multispecific binding moiety of the present disclosure comprises:
a PD-1 binding domain of the present disclosure comprising a heavy chain variable region having an amino acid sequence as set forth in SEQ ID NO: 8, and
a LAG-3 binding domain of the present disclosure comprising a heavy chain variable region having an amino acid sequence as set forth in SEQ ID NO: 17.

In one embodiment, the multispecific binding moiety of the present disclosure comprises:
a PD-1 binding domain of the present disclosure comprising a heavy chain variable region having an amino acid sequence as set forth in SEQ ID NO: 1, and
a LAG-3 binding domain of the present disclosure comprising a heavy chain variable region having an amino acid sequence as set forth in SEQ ID NO: 15;
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 12,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 13,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 14,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 6, and
a LAG-3 binding domain of the present disclosure comprising a heavy chain variable region having an amino acid sequence as set forth in SEQ ID NO: 15,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 5, and
a LAG-3 binding domain of the present disclosure comprising a heavy chain variable region having an amino acid sequence as set forth in SEQ ID NO: 14,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 7, and
a LAG-3 binding domain of the present disclosure comprising a heavy chain variable region having an amino acid sequence as set forth in SEQ ID NO: 17,
wherein the PD-1 binding domain and LAG-3 binding domain comprise a light chain variable region having an amino acid sequence as set forth in SEQ ID NO: 24.

In one embodiment, the multispecific binding moiety of the present disclosure comprises:
a PD-1 binding domain of the present disclosure comprising a heavy chain variable region having an amino acid sequence as set forth in SEQ ID NO: 8, and
a LAG-3 binding domain of the present disclosure comprising a heavy chain variable region having an amino acid sequence as set forth in SEQ ID NO: 17,
wherein the PD-1 binding domain and LAG-3 binding domain comprise a light chain variable region having an amino acid sequence as set forth in SEQ ID NO: 24.

In certain embodiments, a PD-1xLAG-3 multispecific antibody of the present disclosure has higher binding affinity for human PD-1 than a reference anti-human PD-1 antibody comprising two heavy chain variable regions having an amino acid sequence as set forth in SEQ ID NO: 34 and two light chain variable regions having an amino acid sequence as set forth in SEQ ID NO: 35.

Determining if a PD-1xLAG-3 multispecific antibody has a higher binding affinity for human PD-1 than the reference anti-human PD-1 antibody can be done by measuring the binding affinity of both antibodies in the same type of assay, using the same assay conditions. Thus, in certain embodiments, the binding affinity of the PD-1xLAG-3 multispecific antibody and the binding affinity of the reference anti-human PD-1 antibody are measured in the same type of assay, using the same assay conditions. In certain embodiments, the assay is an assay that uses surface plasmon resonance (SPR) to measure binding affinity, such as the biosensor system of Biacore®, or Solution Equilibrium Titration (SET) (see Friguet B et al. (1985) J. Immunol Methods; 77(2): 305-319, and Hanel C et al. (2005) Anal Biochem;

339(1): 182-184).The binding affinity values of the PD-1xLAG-3 multispecific antibodies as provided herein are obtained with the method described in Example 12.

In brief, Example 12 describes determining the binding affinity in bispecific IgG format using SPR on a BlAcore-T200 instrument using an anti-huIgG antibody immobilized on a CMS Series S sensor chip. Monomeric recombinant antigens used are: huLAG-3 (huLAG-3-His, Sino Biological, Cat. nr. 16498-H08H), cyLAG-3 (cyLAG-3-His, Sino Biological, cat. nr. 90841-CO8H), huPD-1 (huPD-1-His, Sino Biological, cat. nr. 10377-H08H) and cyPD-1 (cyPD-1-His, R&D Systems, cat. nr. 8509-PD). Immobilization of goat anti-huIgG Fc on four flow channels of a CMS sensor chip was performed by amine coupling, using 40 μg/ml of the antibody diluted in 10 mM acetate pH 5.0. The following conditions are used: activation time of 420 seconds, deactivation time of 420 seconds, deactivation buffer: 1 M ethanolamine pH 8.5. Density of immobilization ranges from 9158 to 9428 RU. Test and control antibodies are captured by anti-huIgG antibody immobilized on the CM5 sensor chip at a flow rate of 30 μl/min for 60 seconds. Captured antibody concentration is 20 nM for PD-1 affinity determination and 10 nM for LAG-3 affinity determination. This is followed by a stabilization period of 60 seconds with buffer at a flow rate of 30 μl/min. Five step, two fold, serial dilutions of the antigens are injected, at 30 μl/min, for 60 seconds, in both the flow cell with the captured antibody and a reference flow cell (no captured antibody). Antigen concentrations are 80 nM down to 2.5 nM for huPD-1 and cyPD-1, and 40 to 1.25 nM for hu-LAG-3 and cy-LAG-3. Background correction for buffer effects is performed by injection with buffer alone and the reference flow cell is used for background subtraction. Following antibody-antigen interaction, an off-rate wash of 300 seconds, at 30 μl/min is done. Regeneration between cycles is done using two 15 μl injections of 10 mM Glycine pH 1.5 at 30 μl/min, followed by a stabilization step of 90 seconds at 90 μl/min. HBS-EP+buffer is used for PD-1 affinity determination, while, for LAG-3, HBS-EP+ is supplemented with NaCl to a final concentration of 500 mM NaCl. Results are analyzed in Biacore T200 Evaluation Software. The raw RU signal are blank subtracted (channel with no captured antibody) and background corrected for buffer effects (subtraction of the run with captured antibody but with buffer in the second injection, instead of antigen). 1:1 binding Langmuir fitting is applied to the set of sample curves, using the simultaneous fitting option of the Biacore T200 Evaluation Software to calculate association rate (ka), dissociation rate (kd) and affinity (KD). In certain embodiments, the PD-1xLAG-3 multispecific antibody has at least a ten-fold higher binding affinity for human PD-1 than a reference anti-human PD-1 antibody, as measured by SPR as described herein, for instance as described in Example 12. In certain embodiments, the PD-1xLAG-3 multispecific antibody has a ten to fifty fold, preferably a ten to forty, ten to thirty, or ten to twenty, fold higher binding affinity for human PD-1 than the reference anti-human PD-1 binding domain, as measured by SPR as described herein In certain embodiments, the PD-1xLAG-3 multispecific antibody has a ten-fold higher binding affinity for human PD-1 than the reference anti-human PD-1 binding domain, as measured by SPR as described herein, for instance as described in Example 12.

The reference anti-human PD-1 antibody is a nivolumab analog antibody, preferably produced using the same production method as the PD-1xLAG-3 multispecific antibody subject to comparison. A nivolumab analog antibody has the same heavy chain variable region sequence (SEQ ID NO: 20) as nivolumab. A nivolumab analog antibody has the same light chain variable region sequence (SEQ ID NO: 21) as nivolumab.

In certain embodiments, the PD-1xLAG-3 multispecific antibody has a binding affinity for human PD-1 in a range of about 0.1-1.0 nM, in particular in a range of about 0.2-0.4 nM, more in particular in a range of about 0.32-0.34 nM, as measured by SPR as described herein, for instance as described in Example 12.

In certain embodiments, the PD-1xLAG-3 multispecific antibody has a binding affinity for human PD-1 in a range of 0.1-1.0 nM, in particular in a range of 0.2-0.4 nM, more in particular in a range of 0.32-0.34 nM, as measured by SPR as described herein, for instance as described in Example 12.

In certain embodiments, the binding affinity is measured with the PD-1xLAG-3 multispecific antibody of the present disclosure in bivalent bispecific format and the reference anti-human PD-1 antibody in bivalent monospecific IgG format.

The binding affinity for human PD-1 thus represents the monovalent binding affinity of a bivalent bispecific PD-1xLAG-3 antibody.

In certain embodiments, a PD-1xLAG-3 multispecific antibody of the present disclosure has a binding affinity in the range of about 0.4-3.0 nM for cynomolgus PD-1, as measured by surface plasmon resonance (SPR), as described herein, for instance as described in Example 12, in bivalent bispecific antibody format. In certain embodiments, a PD-1xLAG-3 multispecific antibody of the present disclosure has a binding affinity in the range of 0.4-3.0 nM for cynomolgus PD-1, as measured by surface plasmon resonance (SPR), as described herein, for instance as described in Example 12, in bivalent bispecific antibody format. The binding affinity for cynomolgus PD-1 thus represents the monovalent binding affinity of a bispecific PD-1xLAG-3 bivalent antibody.

In certain embodiments, a PD-1xLAG-3 multispecific antibody of the present disclosure has a binding affinity in the range of about 1-2 nM for human LAG-3, as measured by surface plasmon resonance (SPR), as described herein, for instance as described in Example 12, in bivalent bispecific antibody format. In certain embodiments, a PD-1xLAG-3 multispecific antibody of the present disclosure has a binding affinity in the range of 1-2 nM for human LAG-3, as measured by surface plasmon resonance (SPR), as described herein, for instance as described in Example 12, in bivalent bispecific antibody format. The binding affinity of for human LAG-3 thus represents the monovalent binding affinity of a bivalent bispecific PD-1xLAG-3 antibody.

In certain embodiments, a PD-1xLAG-3 multispecific antibody of the present disclosure has a binding affinity in the range of about 0.2-0.4 nM for cynomolgus LAG-3 as measured by surface plasmon resonance (SPR), as described herein, for instance as described in Example 12, in bivalent bispecific antibody format. In certain embodiments, a PD-1xLAG-3 multispecific antibody of the present disclosure has a binding affinity in the range of 0.2-0.4 nM for cynomolgus LAG-3 as measured by surface plasmon resonance (SPR), as described herein, for instance as described in Example 12, in bivalent bispecific antibody format. The binding affinity for cynomolgus PD-1 thus represents the monovalent binding affinity of a bivalent bispecific PD-1xLAG-3 antibody.

In certain embodiments, a multispecific binding moiety of the present disclosure is monovalent for binding to human PD-1, meaning that the multispecific binding moiety comprises only one PD-1 binding domain of the present disclosure. In certain embodiments, multispecific binding moieties of the present disclosure monovalent for binding to PD-1 have a comparable, or equal or higher, binding affinity for PD-1 than bivalent monospecific binding moieties and/or multispecific binding moieties at least bivalent for binding to PD-1 described in the art, at equivalent concentrations. In certain embodiments, such bivalent monospecific binding moiety is a nivolumab analog antibody as described herein. In certain embodiments, multispecific binding moieties of the present disclosure monovalent for binding to PD-1 have a higher potency than a reference multispecific binding moiety, at equivalent concentrations. In certain embodiments, such reference multispecific binding moiety is a nivolumab analog antibody as described herein.

In certain embodiments, further provided herein is a vector useful for producing a multispecific binding moiety of the present disclosure. In certain embodiments, such expression vector comprises a nucleic acid sequence encoding the heavy chain variable region of an anti-human PD-1 binding domain as described herein and a nucleic acid sequence encoding the heavy chain variable region of an anti-human LAG-3 binding domain as described herein. In certain embodiments, a vector of the present disclosure may further comprise a nucleic acid sequence encoding a CHI region and preferably a hinge, CH2 and CH3 region. In certain embodiments, the vector of the present disclosure may further comprise at least one nucleic acid sequence encoding a light chain variable region, and preferably a CL region. In certain embodiments, the light chain variable region can be a common light chain variable region as described herein.

In certain embodiments, the present disclosure also provides a cell comprising a nucleic acid sequence encoding the heavy chain variable region of an anti-human PD-1 binding domain as described herein and a nucleic acid sequence encoding the heavy chain variable region of an anti-human LAG-3 binding domain as described herein. In certain embodiments, a cell of the present disclosure may further comprise a nucleic acid sequence encoding a CH1 region and preferably a hinge, CH2 and CH3 region. In certain embodiments, the cell of the present disclosure may further comprise at least one nucleic acid sequence encoding a light chain variable region, and preferably a CL region. In certain embodiments, the light chain variable region can be a common light chain variable region as described herein.

In certain embodiments, the present disclosure also provides a cell producing a multispecific binding moiety as described herein. In certain embodiments, such cell can be a recombinant cell, which has been transformed with a vector of the present disclosure.

In certain embodiments, further provided herein is a method for producing a variant of a multispecific binding moiety of the present disclosure, wherein the method comprises:
generating a sequence variant of a PD-1 heavy chain variable region and/or LAG-3 heavy chain variable region as described herein; and
expressing the sequence variant(s) and a light chain variable region as described herein in a cell.

Methods for generating sequence variants are well known in the art. One can take a random approach in generating sequence variants or a targeted approach, where one can for instance aim at introducing variations that are likely to increase or decrease binding affinity. Routine methods for affinity maturing antibody binding domains are widely known in the art, see for instance Tabasinezhad M. et al. Immunol Lett. 2019;212:106-113. One can also aim at introducing variations that mitigate developability risks with a view on producing a binding domain, or moiety comprising such binding domain, at large scale. Variations may be introduced that are likely not to cause a loss in binding specificity and/or affect binding affinity. Whether amino acid residues within the CDRs and/or framework regions can be substituted, for instance with a conservative amino acid residue, and without, or substantially without, loss in binding specificity and/or affinity, can be determined by methods well known in the art. Experimental examples include, but are not limited to, for instance, alanine scanning (Cunningham B C, Wells J A. Science. 1989;244(4908):1081-5), and deep mutational scanning (Araya C L, Fowler D M. Trends Biotechnol. 2011;29(9):435-42). Computational methods have also been developed that can predict the effect of amino acid variation, such as for instance described in Sruthi C K, Prakash M. PLoS One. 2020;15(1):e0227621, Choi Y. et al. PLoS One. 2012;7(10):e46688, and Munro D, Singh M. Bioinformatics. 2020;36(22-23):5322-9.

In certain embodiments, further provided herein are any variant multispecific binding moieties, a pharmaceutical composition comprising any variant multispecific binding moieties; nucleic acid encoding a variant binding domain of any of said variant multispecific binding moeities; vectors and cells comprising said nucleic acids; and use of said variant multispecific binding moieties or pharmaceutical composition for the treatment of cancer.

### Pharmaceutical Composition and Methods

A multispecific binding moiety of the disclosure can be used in a pharmaceutical composition, together with a pharmaceutically acceptable carrier, to effectively treat a disease, for example a disease associated with a suppressed immune system, in particular cancer. Treatment includes the administration of an effective amount of the multispecific binding moiety, or pharmaceutical composition, to a subject in need thereof.

In certain embodiments, the present disclosure provides a multispecific binding moiety, or a pharmaceutical composition, as described herein for use in therapy.

In certain embodiments, the present disclosure provides a multispecific binding moiety, or a pharmaceutical composition, as described herein for use in the treatment of a disease associated with a suppressed immune system, in particular cancer.

In certain embodiments, the present disclosure provides a method for treating a disease, wherein the method comprises administering an effective amount of a multispecific binding moiety, or a pharmaceutical composition as described herein to an individual in need thereof.

In certain embodiments, the present disclosure provide a method for treating a disease associated with a suppressed immune system, in particular cancer, wherein the method comprises administering an effective amount of multispecific binding moiety, or a pharmaceutical composition as described herein to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence.

The effective amount of the agent or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual.

An effective amount can be administered in one or more administrations. An effective amount also includes an amount that balances any toxic or detrimental effects of the agent or combination of agents and the therapeutically beneficial effects.

The term “agent” refers to a therapeutically active substance, in the present case a PD-1 binding domain of the present disclosure, a binding moiety (for example a multispecific binding moiety comprising an anti-human PD-1 binding domain) of the present disclosure, or a pharmaceutical composition of the present disclosure.

### General Terms

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. The antigen binding site binds the antigen and a variety of variants thereof, such as those expressed by some antigen positive immune or tumor cells.

When herein reference is made to a gene, a protein, the reference is preferably to the human form of the gene or protein. When herein reference is made to a gene or protein reference is made to the natural gene or protein and to variant forms of the gene or protein as can be detected in tumors, cancers and the like, preferably as can be detected in human tumors, cancers and the like.

HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensemble provides the accession number with which information on the gene or protein encoded by the gene can be obtained from the Ensemble database. Ensembl is a joint project between EMBL-EBI and the Wellcome Trust Sanger Institute to develop a software system which produces and maintains automatic annotation on selected eukaryotic genomes.

### BRIEF DESCRIPTION OF THE DRAWINGS

In the Figures, bivalent monospecific antibodies are indicated in the format SEQ ID NO: A, where SEQ ID NO: A refers to the heavy chain variable sequence of both binding domains. Each binding domain of the monospecific antibodies comprises a light chain. In the Examples, which are used to illustrate the present disclosure but are not intended to limit the disclosure in any way, each binding domain of the monospecific antibodies comprises a light chain variable region having an amino acid sequence as set forth in SEQ ID NO:24 and a light chain constant region having an amino acid sequence as set forth in SEQ ID NO: 71. The monospecific antibodies preferably are IgG1 antibodies comprising a CH1, hinge, CH2, and CH3. In the Examples, which are used to illustrate the present disclosure but are not intended to limit the disclosure in any way, monospecific antibodies were screened in IgG1 format, wherein the PD-1 binding heavy chains comprise a CH1 having an amino acid sequence as set forth in SEQ ID NO: 29, a CH2 having an amino acid sequence as set forth in SEQ ID NO: 72, and a CH3 having an amino acid sequence as set forth in SEQ ID NO: 73.

Bivalent bispecific antibodies are indicated in the format SEQ ID NO: A×SEQ ID NO: B, where both SEQ ID NO: A and B refer to heavy chain variable sequences. Each binding domain of the bispecific antibodies comprises a light chain. In the Examples, which are used to illustrate the present disclosure but are not intended to limit the disclosure in any way, each binding domain of the monospecific antibodies comprises a light chain variable region variable region having an amino acid sequence as set forth in SEQ ID NO:24 and a light chain constant region having an amino acid sequence as set forth in SEQ ID NO: 71. The bispecific antibodies preferably are IgG1 antibodies comprising a CH1, hinge, CH2, and CH3. In the Examples, which are used to illustrate the present disclosure but are not intended to limit the disclosure in any way, bispecific antibodies were screened in IgG1 format, wherein the PD-1 binding heavy chain comprises a CHI having an amino acid sequence as set forth in SEQ ID NO: 29, a CH2 having an amino acid sequence as set forth in SEQ ID NO: 30, and a CH3 having an amino acid sequence as set forth in SEQ ID NO: 31; and the LAG-3 binding heavy chain comprises a CH1 having an amino acid sequence as set forth in SEQ ID NO: 29, a CH2 having an amino acid sequence as set forth in SEQ ID NO: 32, and a CH3 having an amino acid sequence as set forth in SEQ ID NO: 33.

Bivalent monospecific nivolumab and relatlimab analog antibodies are indicated in the format SEQ ID NO: A/SEQ ID NO: B, where SEQ ID NO: A refers to the respective heavy chain sequence and SEQ ID NO: B refers to the respective light chain sequence. Bivalent monospecific nivolumab analog antibodies comprise two PD-1 binding domains. Bivalent monospecific relatlimab analog antibodies comprise two LAG-3 binding domains. A combination of nivolumab and relatlimab analogs is indicated in the format SEQ ID NO: A/SEQ ID NO: B+SEQ ID NO: C/SEQ ID NO: D, where SEQ ID NO: A refers to the heavy chain sequence and SEQ ID NO: B refers to the light chain sequence of either nivolumab or relatlimab analog, and SEQ ID NO: C to the heavy chain sequence and SEQ ID NO: D to the light chain sequence of the other. The nivolumab analog antibody is used in

IgG1 or IgG4 format, and each binding domain comprises a light chain. The relatlimab analog antibody is used in IgG1 format, and each binding domain comprises a light chain.

FIG. 1 shows the results of screening of affinity matured variants in a PD-1/PD-L1 reporter assay. A) IgG' s comprising affinity matured heavy chain variable regions having an amino acid sequence as set forth in SEQ ID NO: 1, SEQ ID NO: 2, and SEQ ID NO: 6; were compared with parental antibody comprising a heavy chain variable region having an amino acid sequence as set forth in SEQ ID NO: 9, a nivolumab analog (SEQ ID NO: 18/SEQ ID NO: 22) as a positive control, and a negative control (SEQ ID NO: 23/SEQ ID NO: 24). B) IgG's comprising affinity matured heavy chain variable regions having an amino acid sequence as set forth in SEQ ID NO: 3, SEQ ID NO: 4, and SEQ ID NO: 5; were compared with parental antibody comprising a heavy chain variable region having an amino acid sequence as set forth in SEQ ID NO: 10, nivolumab analogs (SEQ ID NO: 18/SEQ ID NO: 22 and SEQ ID NO: 19/SEQ ID NO: 22) as positive controls, and a negative control (SEQ ID NO: 23/SEQ ID NO: 24).

FIG. 2 shows the results of screening of bispecific antibodies in a PD-1/PD-L1 reporter assay. A) Bispecific antibodies comprising heavy chain variable regions having an amino acid sequence as set forth in SEQ ID NO: 1 and SEQ ID NO: 11, SEQ ID NO: 6 and SEQ ID NO: 11, SEQ ID NO: 5 and SEQ ID NO: 11; SEQ ID NO: 1 and SEQ ID NO: 12, SEQ ID NO: 6 and SEQ ID NO: 12, and SEQ ID NO: 5 and SEQ ID NO: 12, were compared with a nivolumab analog (SEQ ID NO: 20/SEQ ID NO: 22) as a positive control, and a negative control (SEQ ID NO: 23/SEQ ID NO: 24). B) Bispecific antibodies comprising heavy chain variable regions having an amino acid sequence as set forth in SEQ ID NO: 1 and SEQ ID NO: 13, SEQ ID NO: 6 and SEQ ID NO: 13, SEQ ID NO: 5 and SEQ ID NO: 13; SEQ ID NO: 1 and SEQ ID NO: 14, SEQ ID NO: 6 and SEQ ID NO: 14, and SEQ ID NO: 5 and SEQ ID NO: 14, were compared with a nivolumab analog (SEQ ID NO: 20/SEQ ID NO: 22) as a positive control, and a negative control (SEQ ID NO: 23/SEQ ID NO: 24). C) Bispecific antibodies comprising heavy chain variable regions having an amino acid sequence as set forth in SEQ ID NO: 1 and SEQ ID NO: 15, SEQ ID NO: 6 and SEQ ID NO: 15, SEQ ID NO: 5 and SEQ ID NO: 15; SEQ ID NO: 1 and SEQ ID NO: 16, SEQ ID NO: 6 and SEQ ID NO: 16, and SEQ ID NO: 5 and SEQ ID NO: 16, were compared with a nivolumab analog (SEQ ID NO: 20/SEQ ID NO: 22) as a positive control, and a negative control (SEQ ID NO: 23/SEQ ID NO: 24).

FIG. 3 shows the results of screening of bispecific antibodies in a PD-1/PD-L1 reporter assay. A) Comparison of potencies of bispecific antibodies comprising heavy chain variable regions having an amino acid sequence as set forth in SEQ ID NO: 1, SEQ ID NO: 6, or SEQ ID NO: 5, compared with nivolumab analog 3 (nivolumab*). B) Average EC50 values of bispecific antibodies comprising heavy chain variable regions having an amino acid sequence as set forth in SEQ ID NO: 7, or SEQ ID NO: 8, compared with nivolumab analog 4 (nivolumab*).

FIG. 4 shows the binding affinity of the PD-1 binding domains comprising a heavy chain variable region having an amino acid sequence as set forth in SEQ ID NO: 1, SEQ ID NO: 6, or SEQ ID NO: 5, in bivalent monospecific format, compared with bivalent monospecific nivolumab analog 1 (SEQ ID NO: 18/SEQ ID NO: 22; in quadruplicate) and the PD-1 binding domain of nivolumab analog 1 as part of a bivalent bispecific antibody (SEQ ID NO: 18/SEQ ID NO: 22 x SEQ ID NO: 23/SEQ ID NO: 24).

FIG. 5 shows the results of screening of bispecific antibodies in a PD-1/LAG-3 reporter assay. A) Bispecific antibodies comprising heavy chain variable regions having an amino acid sequence as set forth in SEQ ID NO: 1 and SEQ ID NO: 11, SEQ ID NO: 1 and SEQ ID NO: 12, SEQ ID NO: 1 and SEQ ID NO: 13; SEQ ID NO: 1 and SEQ ID NO: 14, SEQ ID NO: 1 and SEQ ID NO: 15, and SEQ ID NO: 1 and SEQ ID NO: 16, were compared with a nivolumab analog (SEQ ID NO: 20/SEQ ID NO: 22) as a positive control, a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24). B) Bispecific antibodies comprising heavy chain variable regions having an amino acid sequence as set forth in SEQ ID NO: 6 and SEQ ID NO: 11, SEQ ID NO: 6 and SEQ ID NO: 12, SEQ ID NO: 6 and SEQ ID NO: 13; SEQ ID NO: 6 and SEQ ID NO: 14, SEQ ID NO: 6 and SEQ ID NO: 15, and SEQ ID NO: 6 and SEQ ID NO: 16, were compared with a combination of a nivolumab analog and relatlimab analog (SEQ ID NO: 20/SEQ ID NO: 22+SEQ ID NO: 27/SEQ ID NO: 28), and a bivalent monospecific antibody comprising SEQ ID NO: 23/SEQ ID NO: 24 and a motavizumab analog (SEQ ID NO: 25/SEQ ID NO: 26) as negative controls. C) Bispecific antibodies comprising heavy chain variable regions having an amino acid sequence as set forth in SEQ ID NO: 5 and SEQ ID NO: 11, SEQ ID NO: 5 and SEQ ID NO: 12, SEQ ID NO: 5 and SEQ ID NO: 13; SEQ ID NO: 5 and SEQ ID NO: 14, SEQ ID NO: 5 and SEQ ID NO: 15, and SEQ ID NO: 5 and SEQ ID NO: 16, were compared with a combination of a nivolumab analog and relatlimab analog (SEQ ID NO: 20/SEQ ID NO: 22 +SEQ ID NO: 27/SEQ ID NO: 28), and a negative control (SEQ ID NO: 23/SEQ ID NO: 24).

FIG. 6 shows the results of screening of bispecific antibodies in a SEB assay. A) Bispecific antibodies comprising heavy chain variable regions having an amino acid sequence as set forth in SEQ ID NO: 1 and SEQ ID NO: 11, SEQ ID NO: 1 and SEQ ID NO: 12, SEQ ID NO: 1 and SEQ ID NO: 13; SEQ ID NO: 1 and SEQ ID NO: 14, SEQ ID NO: 1 and SEQ ID NO: 15, and SEQ ID NO: 1 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24). B) Bispecific antibodies comprising heavy chain variable regions having an amino acid sequence as set forth in SEQ ID NO: 6 and SEQ ID NO: 11, SEQ ID NO: 6 and SEQ ID NO: 12, SEQ ID NO: 6 and SEQ ID NO: 13; SEQ ID NO: 6 and SEQ ID NO: 14, SEQ ID NO: 6 and SEQ ID NO: 15, and SEQ ID NO: 6 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24). C) Bispecific antibodies comprising heavy chain variable regions having an amino acid sequence as set forth in SEQ ID NO: 5 and SEQ ID NO: 11, SEQ ID NO: 5 and SEQ ID NO: 12, SEQ ID NO: 5 and SEQ ID NO: 13; SEQ ID NO: 5 and SEQ ID NO: 14, SEQ ID NO: 5 and SEQ ID NO: 15, and SEQ ID NO: 5 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24).

FIG. 7 shows the results of screening of bispecific antibodies in an antigen recall assay. A) Bispecific antibodies comprising heavy chain variable regions having an amino acid sequence as set forth in SEQ ID NO: 1 and SEQ ID NO: 11, SEQ ID NO: 1 and SEQ ID NO: 12, SEQ ID NO: 1 and SEQ ID NO: 13; SEQ ID NO: 1 and SEQ ID NO: 14, SEQ ID NO: 1 and SEQ ID NO: 15, and SEQ ID NO: 1 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24). B) Bispecific antibodies comprising heavy chain variable regions having an amino acid sequence as set forth in SEQ ID NO: 6 and SEQ ID NO: 11, SEQ ID NO: 6 and SEQ ID NO: 12, SEQ ID NO: 6 and SEQ ID NO: 13; SEQ ID NO: 6 and SEQ ID NO: 14, SEQ ID NO: 6 and SEQ ID NO: 15, and SEQ ID NO: 6 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24). C) Bispecific antibodies comprising heavy chain variable regions having an amino acid sequence as set forth in SEQ ID NO: 5 and SEQ ID NO: 11, SEQ ID NO: 5 and SEQ ID NO: 12, SEQ ID NO: 5 and SEQ ID NO: 13; SEQ ID NO: 5 and SEQ ID NO: 14, SEQ ID NO: 5 and SEQ ID NO: 15, and SEQ ID NO: 5 and SEQ ID NO: 16, were compared with a combination of a nivolumab analog and relatlimab analog (SEQ ID NO: 20/SEQ ID NO: 22+SEQ ID NO: 27/SEQ ID NO: 28), and a negative control (SEQ ID NO: 23/SEQ ID NO: 24).

FIG. 8 shows the efficacy of bispecific antibodies in an in vivo mouse study. A) Efficacy of bispecific antibody 1 (10 mg/kg), bispecific antibody 2 (10 mg/kg), and bispecific antibody 3 (10 mg/kg) (FIG. 8A), and bispecific antibody 4 (10 mg/kg) and bispecific antibody 5 (10 mg/kg) (FIG. 8B), to reduce tumor volume was compared with an IgG1 control antibody (10 mg/kg), IgG4 control antibody (10 mg/kg), pembrolizumab (10 mg/kg), relatlimab analog (10 mg/kg), and a combination of pembrolizumab (10 mg/kg) and relatlimab analog(10 mg/kg). C) Percentage of regulatory T cells (Treg) measured in tumors obtained from mice treated with bispecific antibody 1, bispecific antibody 2, or bispecific antibody 3, compared with an IgG1 control antibody, IgG4 control antibody, pembrolizumab, relatlimab analog, and a combination of pembrolizumab and relatlimab analog. D) Percentage of CD8⁺ T cells (left graph) and ratio of regulatory T cells within this CD8⁺ T cell population (right graph), measured in tumors obtained from mice treated with bispecific antibody 1, bispecific antibody 2, or bispecific antibody 3, compared with an IgG1 control antibody, IgG4 control antibody, pembrolizumab, relatlimab analog, and a combination of pembrolizumab and relatlimab analog.

FIG. 9 shows the binding affinity of bispecific antibodies comprising SEQ ID NO: 7 and SEQ ID NO: 17, and SEQ ID NO: 8 and SEQ ID NO: 17 to human and cynomolgus PD-1 and LAG-3, compared with a nivolumab analog (SEQ ID NO: 21/SEQ ID NO: 22) and a relatlimab analog (SEQ ID NO: 27/SEQ ID NO: 28).

The following Examples illustrate the present disclosure but are not intended to limit the disclosure in any way.

### EXAMPLES

### Example 1

### Generation of Anti-Human PD-1 Binding Domains

Anti-human PD-1 binding domains can be obtained by methods known in the art, such as for instance as described in WO 2019/009728. A large panel of heavy chain variable regions were obtained by immunizing transgenic mice comprising a common IGKV1-39 light chain (MeMo® mice) with human PD-1 antigenic moieties, including the use of different forms of DNA, protein and cell-based antigen delivery. Heavy chain variable regions of SEQ ID NO: 9 and SEQ ID NO: 10 were selected for affinity maturation. This resulted in 202 affinity matured variants of which a number were selected for further characterization in a PD-1/PD-L1 reporter assay.

### Example 2

### Potency of PD-1 IgGs and Selection for Production of Bispecific Abs

In order to confirm that the affinity matured PD-1 heavy chain variable regions in IgG format are at least as potent as their parental IgGs, affinity matured variants were screened in a PD-1/PD-L1 reporter assay. Also included in the assay were the parental anti-PD-1 IgGs, an anti-PD-1 antibody comprising the heavy chain (SEQ ID NO: 18) and light chain (SEQ ID NO: 22) of nivolumab (Fc-silenced IgG1 nivolumab analog 1), and an anti-PD-1 antibody comprising the heavy chain (SEQ ID NO: 19) and light chain (SEQ ID NO: 22) of nivolumab (IgG4 nivolumab analog 2) as positive controls, and an anti RSV-G antibody comprising the heavy chain variable region having SEQ ID NO: 23 and light chain variable region having SEQ ID NO: 24 as a negative control. The last 2 wells in this column were left without IgG as a basal level control.

The PD-1-PD-L1 reporter assay was performed according to manufacturer's protocol (Promega, cat. no. J1255), which uses two cell lines—PD-L1 aAPC/CHO-K1 cells which are CHO-K1 cells expressing human PD-L1 and an engineered cell surface protein designed to activate cognate TCRs in an antigen-independent manner (Promega, cat. no. J109A); and PD-1 effector cells: Jurkat T cells expressing human PD-1 and a luciferase reporter driven by an NFAT response element (NFAT-RE) (Promega, cat. no. J115A).

On day 1, Cell Recovery Medium for PD-L1 cells was prepared at room temperature: 10% FBS (Sigma, cat. no. F2442) in DMEM/F12 (Life Technologies, cat. no. 21765). The required number of PD-L1 cell vials (J109A; 1 vial per 32 IgGs to be tested) were removed from the freezer, thawed quickly at 37° C. and cells transferred to a 50 ml tube. Cell Recovery Medium was slowly added to cells, 14.5 ml/vial , volume doubling per minute. Wells of ½-area plates (Corning, cat. no. 3688) were filled with this cell suspension at 50 μl/wellor with 50 μl PBS (Invitrogen, cat. no. 10010). Assay plates were incubated overnight at 37° C., 5% CO2 and 95% Relative Humidity.

On the second day, 2× concentrated Assay Buffer was prepared: 4% FBS (Sigma, cat. no. F2442) in RPMI 1640 (Promega kit or Life Technologies, cat. no. 21875) at room temperature. 2× concentrated test and control IgG solutions were prepared in PBS. Serial dilutions of test and control IgGs were also made in PBS in U-bottom plates (Nunc, cat. no. 268152), starting with 10 μg/ml and performing 6-step 4-fold titration. Positive and negative control IgG serial dilutions were prepared in PBS on separate deep well plates (Greiner Bio-one, cat. no. 780270). Basal control, which is control without IgG was also prepared. IgGs of which activities need to be compared directly were incubated on same plate as much as possible, to avoid inter-plate variation.

Assay plates were taken out of the incubator and flicked to empty wells. 20 μl of IgG solution was added to assay plate, starting with transfer of lowest IgG concentration followed by higher concentration with same pipet tips.

Required number of PD-1 effector cells (J115A: 1 vial per 32 IgGs to be tested) were removed from freezer, thawed quickly at 37° C. and gently mixed by pipetting up and down. Cells from all vials were transferred to a 50 ml tube. 2× concentrated Assay Buffer (5.9 ml per vial of cells) was slowly added to cells such that volume doubled per minute. 20 μl of effector cell suspension was added to wells on assay plates. Plates were incubated for 6 hours at 37° C., 5% CO2 and 95% Relative Humidity. Following 6 hours incubation, plates were pre-incubated at room temperature for 10 min.

Luciferase activity was measured using the Bio-Glo™ luciferase Assay System (Promega, cat. no. G7941). Bio-Glo™ Luciferase Assay Buffer (protected from light) was equilibrated to room temperature overnight and thoroughly mixed with Bio-Glo™ Luciferase

Assay Substrate. 40 μl of Bio-Glo luciferase was added to each well on the assay plate and luminescence measured after 5-10 min on EnVision plate reader (PerkinElmer, Model 2104-0040A Luminescence mode). Readout was obtained in Relative light unit (RLU) values. Fold Induction which is ratio of experimental activity to control activity was calculated as RLU value of IgG-X/RLU value of no IgG. Fold Induction was plotted against log IgG concentrations and the sigmoid curve fitted using GraphPad Prism using non-linear regression and the log(inhibitor) vs. response (three parameters) equation.

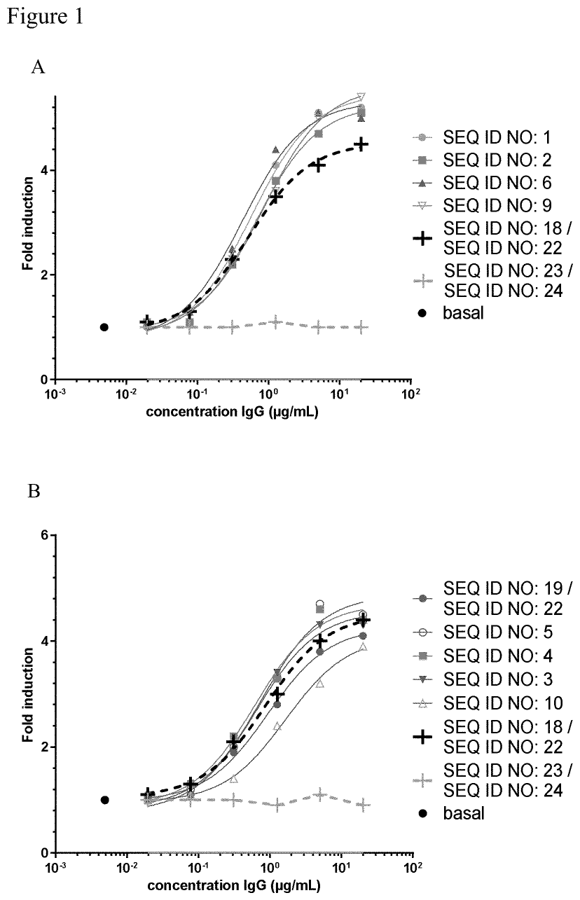

Results are shown in FIG. 1. All controls displayed the expected activities and were consistent in different plates. The affinity matured variants were at least as potent as their parental IgG, and as potent or more potent than nivolumab analog 1. EC50 values of the affinity matured variants and parental antibodies are shown in Table 2.

TABLE 2
EC50 values of affinity matured variants and parental antibodies.

| IgG comprising a | | EC50 (nM) |
|---|---|---|
| VH having amino | | nivolumab |
| acid sequence: | EC50 (nM) | analog |
| SEQ ID NO: 1 | 3.81 | 3.47 |
| SEQ ID NO: 2 | 4.49 | |
| SEQ ID NO: 6 | 2.87 | |
| SEQ ID NO: 9 | 5.65 | |
| SEQ ID NO: 5 | 4.91 | 5.79 |
| SEQ ID NO: 4 | 4.12 | |
| SEQ ID NO: 3 | 4.20 | |
| SEQ ID NO: 10 | 11.05 | |

Three affinity matured PD-1 variants were selected for the generation of bispecific antibodies, wherein the human-PD-1 binding arm is combined with a human-LAG-3 binding arm. These three PD-1 variants comprise a heavy chain variable region having an amino acid sequence as set forth in SEQ ID NO: 1; 5; and 6, and were combined with six different anti-human LAG-3 binding arms. The six anti-human LAG-3 binding arms comprise a heavy chain variable region having an amino acid sequence as set forth in SEQ ID NO: 11, 12, 13, 14, 15, and 16. The resulting eighteen bispecific antibodies were tested for binding to human PD-1 and human LAG-3 using FACS.

### Example 3

### FACS Analysis

The binding of bispecific antibodies was analyzed by FACS using cell lines stably transfected with human LAG-3 or rhesus LAG-3, or transiently transfected with human PD-1 or cynomolgus PD-1. To this end, 293FF cells were transiently transfected with pVAX expression constructs encoding human PD-1 and cynomolgus PD-1, and 293FF cells were stably transfected with pVAX expression constructs encoding human LAG-3 and rhesus LAG-3. IgG specific binding was measured by FACS using a 8-step, 5-fold dilution starting at 50 ug/ml. A goat-anti-human PE was used as secondary detection antibody. Cells w/o staining or only secondary detection antibody were included as negative assay controls. A bivalent monospecific PD-1 antibody comprising heavy chains having SEQ ID NO: 18 and light chains having SEQ ID NO: 22 (nivolumab analog 1), and a bivalent monospecific LAG-3 antibody comprising heavy chains having SEQ ID NO: 27 and light chains having SEQ ID NO: 28 (25F7/relatlimab analog), known to bind PD-1 and LAG-3 in this assay, respectively, were used as a positive control. A bivalent monospecific RSV-G antibody comprising heavy chain variable regions having SEQ ID NO: 23 and light chain variable region having SEQ ID NO: 24 (Fc-silenced IgG1 isotype control) was used as a negative control.

Positive and negative controls behaved as expected. All bispecific antibodies bound to human LAG-3 and rhesus LAG-3. All bispecific antibodies also bound to human PD-1 and cynomolgus PD-1.

### Example 4

### PD-1/PD-L1 Reporter Assay

Bispecific antibodies were screened in a PD-1/PD-L1 reporter assay following the protocol as described in Example 2.

The bispecific antibodies were 6-fold diluted in 6 steps starting at 100 ug/ml final concentration and tested in duplicate. IgG dilutions were prepared in PBS. As a positive control, a 6-step 6-fold titration of a bivalent monospecific PD-1 antibody comprising heavy chains having an amino acid sequence as set forth in SEQ ID NO: 20 and light chains having SEQ ID NO: 22 (IgG4 nivolumab analog 3), starting at 100 μg/ml (final concentration), was included. As an isotype control for the PD-1xLAG-3 bispecific antibodies, a 4-step 6-fold titration of a bivalent monospecific antibody binding to RSV-G, comprising heavy chain variable regions having an amino acid sequence as set forth in SEQ ID NO: 23 and light chain variable regions having SEQ ID NO: 24 (Fc-silenced IgG1 isotype control), was used starting at 100 μg/ml (final concentration). The last two wells in the isotype columns were left without IgG as a basal level control. Antibodies were incubated for 6 hours at 37° C., 5% CO2, 95% RH. Bio-Glo luciferase was added and luminescence was measured on an Envision plate reader (PerkinElmer). Fold induction induced by each antibody was calculated relative to wells containing no antibody (basal).

If an antibody blocks the PD-1/PD-L1 axis, this relieves the inhibition of the TCR signal. The TCR signal then becomes active, and leads to gene transcription and luciferase activity. Luciferase activity correlates with antibody binding and axis blocking.

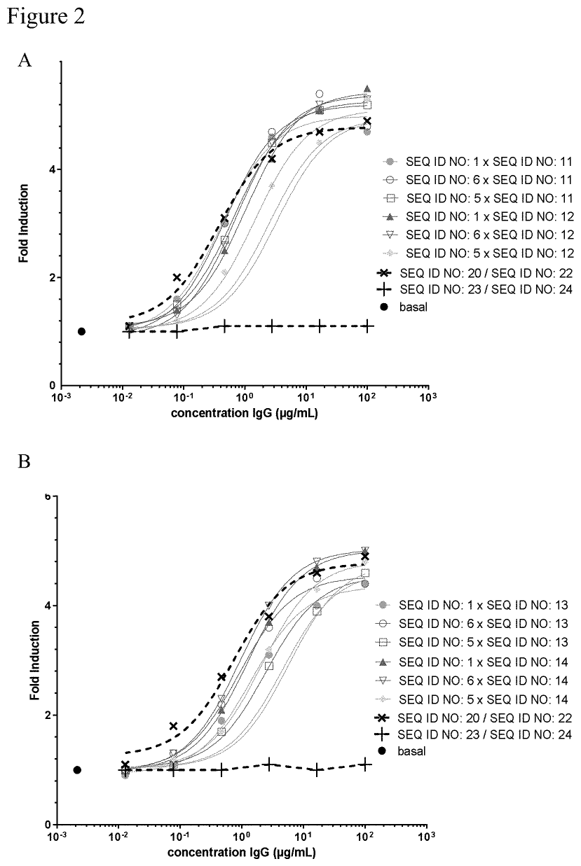

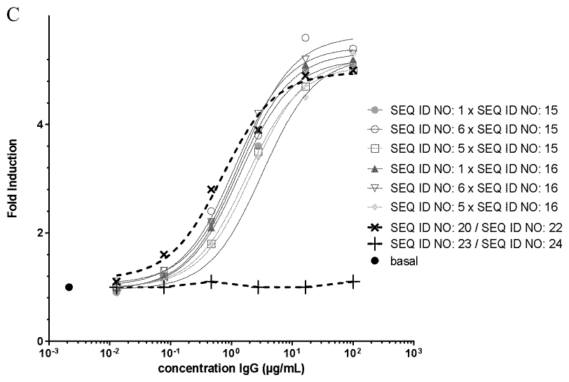

Results are shown in FIG. 2. Positive and negative controls behave as expected. All bispecific antibodies show blocking of the PD-1/PD-L1 axis.

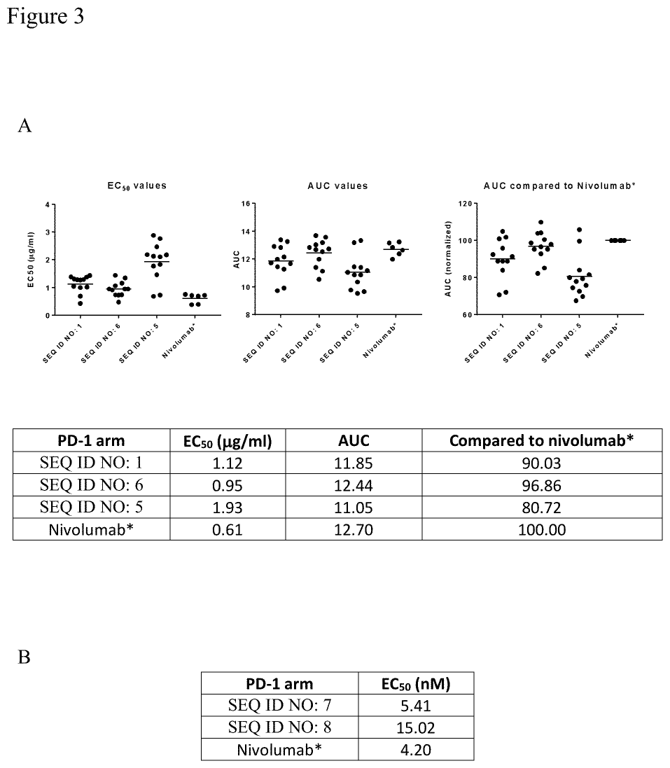

FIG. 3A shows a comparison of potencies in a luciferase reporter assay between three bispecific antibodies and the PD-1 reference antibody nivolumab analog 3. The potency of a bispecific antibody comprising a PD-1 binding domain with a heavy chain variable region having an amino acid sequence as set forth in SEQ ID NO: 6 is similar to the potency of the reference PD-1 antibody. This bispecific antibody thus achieves a similar potency with a single PD-1 binding domain as the reference antibody which is bivalent for binding to PD-1.

The potency of two further bispecific antibodies was also assessed in a PD-1/PD-L1 reporter assay, and compared with a bivalent monospecific PD-1 antibody comprising heavy chains having an amino acid sequence as set forth in SEQ ID NO: 21 and light chains having SEQ ID NO: 22 (nivolumab analog 4). The assay was repeated twice with each sample in triplicate. The average EC50 values are provided in FIG. 3B.

PD-1 binding domains having SEQ ID Nos: 1, 5, and 6 were tested by SPR in bivalent monospecific format to determine binding affinity to PD-1, and compared with an analog of reference antibody nivolumab. Previous studies indicated that the binding affinity to PD-1 is similar for these PD-1 binding domains in bivalent monospecific format and bispecific format. The binding affinity of the nivolumab analog was determined in bivalent monospecific format and bispecific format monovalent for PD-1 (PD-1xRSV).

SPR experiments were performed using a Biacore 8K instrument (GE Healthcare) at 25° C. The SPR running buffer (10 mM HEPES, 150 mM NaCl, 3 mM EDTA and 0.05% v/v Surfactant P20, pH 7.4) was prepared from 10× HBS-EP Buffer (GE Healthcare). Anti-human Fc antibodies (GE Healthcare) were immobilized via amine coupling on all sixteen flow cells of an S series sensor chip CMS (GE Healthcare). The immobilization levels are ˜9000 RU for all flow cells. The desired capturing level (100-150 RU) of anti-PD-1 antibodies was achieved by flowing appropriate concentration of anti-PD-1 antibodies through the active flow cell of each channel for 60 seconds with 10 μL/min flow rate. Then, a PD-1 three-fold serial dilution concentration series (total 7 concentrations, highest at 300 nM) prepared from PD-1 stock (R&D 8986-PD) and running buffer (0 concentration) were injected for 240 seconds (association time) immediately followed by running buffer for 480 seconds (dissociation time) at a flow rate of 45 μL/min. Surface was regenerated with 30-second injection of 3 M MgCl₂ with 30 μL/min flow rate. Binding kinetics and affinity parameters were obtained from a global fit of the data to 1 to 1 binding model.

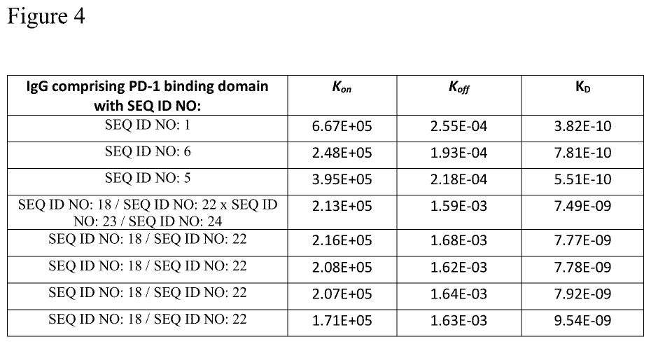

Results are presented in FIG. 4. The PD-1 binding domains of the three bispecific antibodies have at least a ten-fold higher binding affinity for PD-1 than the nivolumab analog in both antibody formats.

### Example 5

### PD-1/LAG-3 Reporter Assay

PD-L1 Raji cells (Promega, cat. no. CS1978B03) were prepared by suspending the cells in assay medium (1% hiFBS (Gibco, Gibco/Thermo Fisher, cat. no. 10270106) in RPMI 1640 (+25 mM HEPES) (Life Technologies, cat. no. 52400) at room temperature) to arrive at 2 million cells/ml. Jurkat PD-1 and LAG-3 effector cells (Promega, cat. no. CS1978B02) were prepared by suspending the cells in assay medium to arrive at 4 million cells/ml. 3× concentrated test and control IgG solutions were prepared in PBS, i.e. by a 6-fold serial dilution starting between 6-300 μg/ml, with a dilution factor between 2 and 10 (final assay concentration starting between 20-100 μg/ml).

Since this assay can be very sensitive to the batch of FBS used, the batch of FBS should be validated prior to performing the assay.

Assay plates were filled with 25 μl Jurkat PD-1 and LAG-3 effector cells or PBS. 25 μl of test and control IgG solution was added. IgGs of which activities need to be compared directly should be incubated on same assay plate as much as possible, to avoid influence of inter-plate variation (plate effects).

An equal volume of PD-L1 Raji cell suspension was mixed with the same volume of SED solution (100 ng/ml of Staphylococcal enterotoxin D (Toxin Technologies, cat. no. PD303) in assay medium). 25 μl of Raji/SED mix was added to the assay plates.

Assay plates were incubated for 6 hours at 37° C., 5% CO2 and 95% Relative Humidity.

After 6 hours of incubation, assay plates were left at room temperature for 10 minutes. 75 μl of Steady-Glo luciferase (Promega, cat. no. E2510) was added to the wells and Luminescence measured after 5-10 minutes on Envision plate reader (according to the Luminescence protocol; PerkinElmer, Model 2104-0020A).

Fold induction induced by each antibody was calculated relative to wells containing no IgG.

IgG's were compared with a positive control, and with a combination of bivalent monospecific PD-1 antibody nivolumab analog 3 and bivalent monospecific LAG-3 antibody 25F7/relatlimab analog, with a highest concentration 50 μg/ml+50 μg/ml. All IgGs were tested in triplicate.

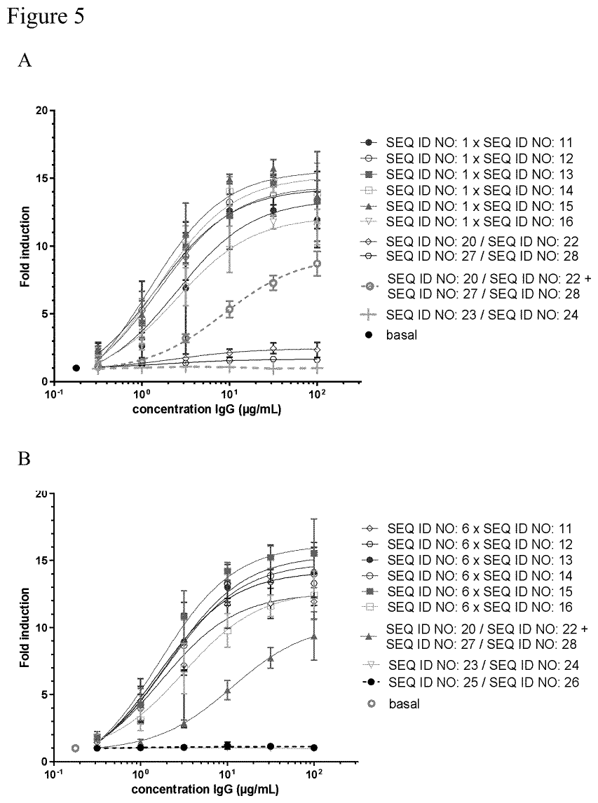

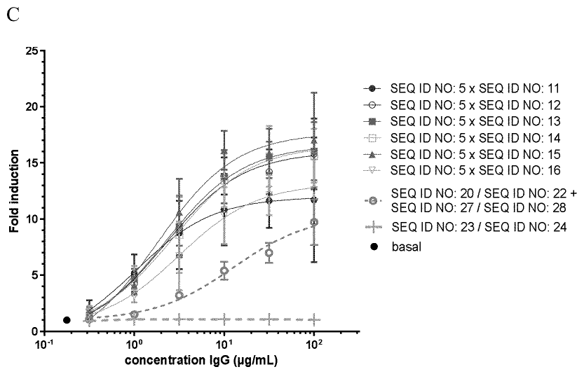

Results are shown in FIG. 5. Positive and negative controls behaved as expected. All bispecific antibodies were more potent in lifting the inhibitory activity of the PD-1 and LAG-3 pathways than the combination of PD-1 and LAG-3 reference antibodies. Percentage of area under the curve (AUC) relative to positive control and EC50 values are provided in Table 3.

### Example 6

### SEB Assay

IgG's were tested in a 6-step 7-fold dilution titration starting at 50 μg/ml (final concentration). IgG dilutions were prepared at 4× final concentration in assay medium. As a positive control, a combination of bivalent monospecific PD-1 antibody nivolumab analog 3 and bivalent monospecific LAG-3 antibody 25F7/relatlimab analog, with a highest concentration 25 μg/ml+25 μg/ml, was included. All IgG's were tested in triplicate.

About 200.000 cryopreserved PBMCs known to respond to anti-LAG-3 and anti-PD-1/PD-L1 IgG were incubated with the bispecific and control antibodies and SEB in a final concentration of 2 ug/ml for 3 days at 37° C., 5% CO2, and 90% RH. After 3 days, the supernatant was harvested, diluted 4-fold, and IL-2 levels were measured with Luminex.

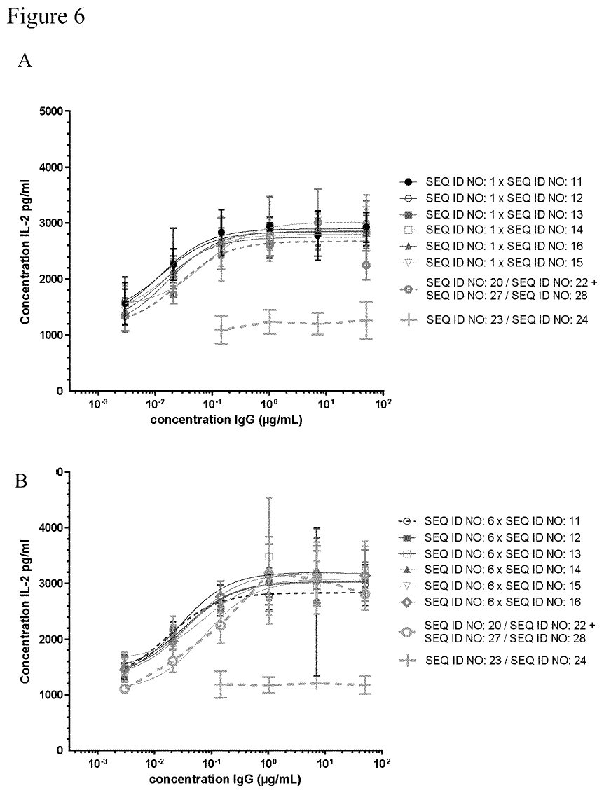

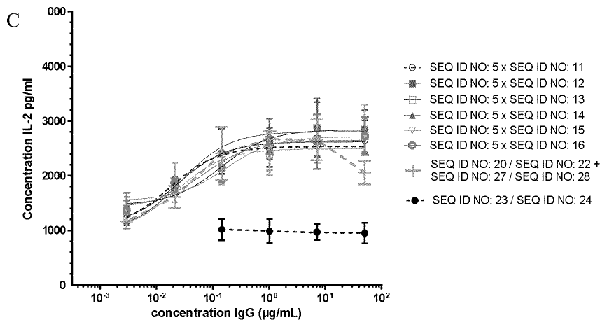

The assay was performed with PBMC's from two donors. The data from one donor is shown in FIG. 6. Positive and negative controls behaved as expected. Many of the bispecific antibodies induced IL-2 release more effectively than the combination of PD-1 and LAG-3 reference antibodies. Percentage of area under the curve (AUC) values relative to positive control are provided in Table 3.

### Example 7

### Antigen Recall Assay

IgG's were tested in a 6-step 5-fold dilution titration starting at 10 μg/ml (final concentration). As a positive control, a combination of bivalent monospecific PD-1 antibody nivolumab analog 3 and bivalent monospecific LAG-3 antibody 25F7/relatlimab analog, with a highest concentration 5 μg/ml+5 μg/ml, was included. As a negative control, a 4-step 5-fold dilution of an antibody against RSV-G comprising heavy chain variable regions having SEQ ID NO: 23 and light chain variable regions having SEQ ID NO: 24 (Fc-silenced IgG1 isotype control) was used starting at 10 μg/ml. Two wells were left without peptide pool as a negative control. All IgG's were tested in triplicate.

About 300.000 PBMC's from a selected donor and rested overnight were incubated with the IgG's and CEFT MHC-II CD4 peptides in a final concentration of 1 ug/ml for 6 days at 37° C., 5% CO2, and 90% RH. The supernatant was harvested to measure the levels of IFN-γ and TNF-α with Luminex.

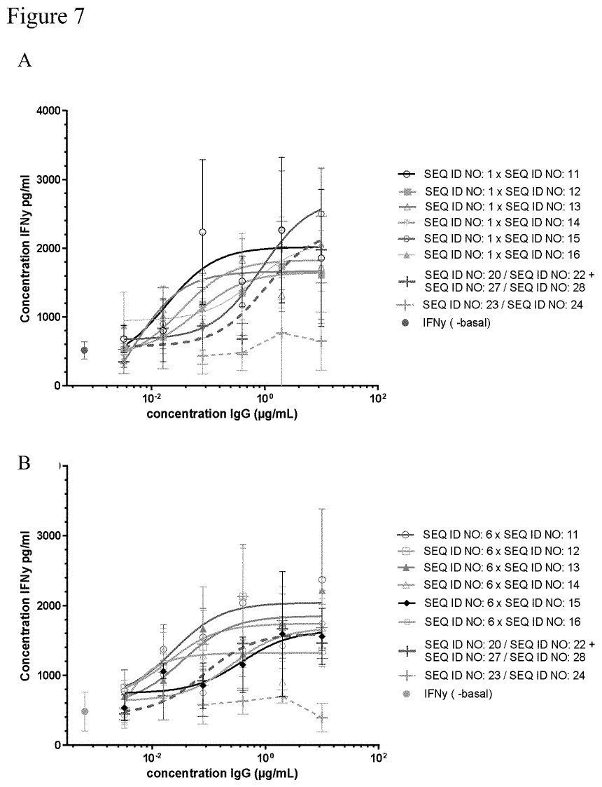

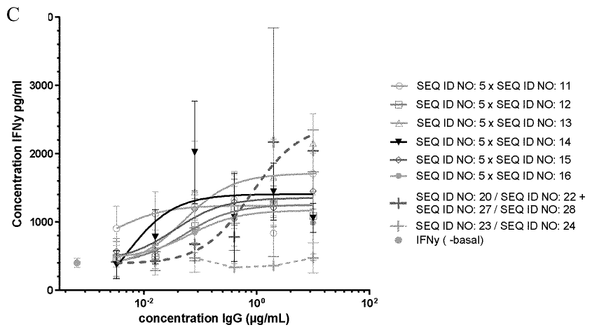

Results are shown in FIG. 7. Positive and negative controls behaved as expected. Many of the bispecific antibodies induced IFN-γ release more effectively than the combination of PD-1 and LAG-3 reference antibodies. The results of the TNF-α readout were similar to those for IFN-y. Percentage of area under the curve (AUC) values relative to positive control are provided in Table 3.

TABLE 3
EC50 and percentage of AUC values relative to positive
control for the bispecific antibodies screened in the
PD-1/LAG-3 reporter assay, SEB assay, and antigen recall assay.

| | | AUC relative | | | |
|---|---|---|---|---|---|
| | | to positive | AUC relative | AUC relative to | |
| | EC50 (nM) | control | to positive | positive control | |
| Bispecific | PD-l/LAG-3 | PD-l/LAG-3 | control | Antigen recall assay | |
| antibody | reporter assay | reporter assay | SEB assay | TNF α | IFNγ |
| SEQ ID NO: 1 × | 17.65 | 194.1 | 109.11 | 142.30 | 199.49 |
| SEQ ID NO: 16 | | | | | |
| SEQ ID NO: 1 × | 8.00 | 289.8 | 106.12 | 140.39 | 171.30 |
| SEQ ID NO: 15 | | | | | |
| SEQ ID NO: 1 × | 9.96 | 271.3 | 108.89 | 148.39 | 182.43 |
| SEQ ID NO: 14 | | | | | |
| SEQ ID NO: 1 × | 10.45 | 256.6 | 109.58 | 149.55 | 215.80 |
| SEQ ID NO: 13 | | | | | |
| SEQ ID NO: 1 × | 10.47 | 259.4 | 112.69 | 117.25 | 145.13 |
| SEQ ID NO: 12 | | | | | |
| SEQ ID NO: 1 × | 19.10 | 215.3 | 130.05 | 188.98 | 233.75 |
| SEQ ID NO: 11 | | | | | |
| SEQ ID NO: 6 × | 29.04 | 193.3 | 138.48 | 96.69 | 163.30 |
| SEQ ID NO: 16 | | | | | |
| SEQ ID NO: 6 × | 12.45 | 276.3 | 131.32 | 99.50 | 148.40 |
| SEQ ID NO: 15 | | | | | |
| SEQ ID NO: 6 × | 17.98 | 246.0 | 145.38 | 106.67 | 164.42 |
| SEQ ID NO: 14 | | | | | |
| SEQ ID NO: 6 × | 17.46 | 255.2 | 146.55 | 148.45 | 220.03 |
| SEQ ID NO: 13 | | | | | |
| SEQ ID NO: 6 × | 14.80 | 243.4 | 147.49 | 164.84 | 248.72 |
| SEQ ID NO: 12 | | | | | |
| SEQ ID NO: 6 × | 21.26 | 215.4 | 128.49 | 188.31 | 283.49 |
| SEQ ID NO: 11 | | | | | |
| SEQ ID NO: 5 × | 25.35 | 196.7 | 107.50 | 76.28 | 112.08 |
| SEQ ID NO: 16 | | | | | |
| SEQ ID NO: 5 × | 12.89 | 292.7 | 108.50 | 99.37 | 140.39 |
| SEQ ID NO: 15 | | | | | |
| SEQ ID NO: 5 × | 19.76 | 262.6 | 130.52 | 123.36 | 117.94 |
| SEQ ID NO: 14 | | | | | |
| SEQ ID NO: 5 × | 16.06 | 270.5 | 114.93 | 123.49 | 123.67 |
| SEQ ID NO: 13 | | | | | |
| SEQ ID NO: 5 × | 14.66 | 259.6 | 114.64 | 82.22 | 99.02 |
| SEQ ID NO: 12 | | | | | |
| SEQ ID NO: 5 × | 10.14 | 217.9 | 104.38 | 116.30 | 173.95 |
| SEQ ID NO: 11 | | | | | |

### Example 8

### In Vivo Study

Efficacy of five bispecific PD-1xLAG-3 antibodies was evaluated in hu-CD34 mice bearing MDA-MB-231 tumors.

Humanized CD34⁺ NSG mice (Jackson Laboratories) were inoculated subcutaneously with a total of 3×10⁶ MDA-MB-231 tumor cells suspended in 100 μl of serum-free culture medium and matrigel matrix (Corning) in equal volumes. When tumors reached approximately 80-100 mm³, mice were randomized into eight groups with ten mice per group and dosed in 1 × PBS (Life Technologies): 1) IgG1 (10mg/kg); 2) IgG4 (10mg/kg); 3) pembrolizumab (10mg/kg); 4) relatlimab analog (10mg/kg); 5) pembrolizumab (10mg/kg)+relatlimab analog (10mg/kg); 6) PD-1xLAG-3 bispecific antibody 1 (10mg/kg); 7) PD-1xLAG-3 bispecific antibody 2 (10mg/kg); and 8) PD-1xLAG-3 bispecific antibody 3 (10mg/kg), and in a separate experiment: 9) PD-1xLAG-3 bispecific antibody 4 (10mg/kg); and 10) PD-1xLAG-3 bispecific antibody 5 (10mg/kg).

PD-1xLAG-3 bispecific antibody 1 comprises a heavy chain variable region having an amino acid sequence as set forth in SEQ ID NO: 6 (PD-1) and a heavy chain variable region having an amino acid sequence as set forth in SEQ ID NO: 13 (LAG-3).

PD-1xLAG-3 bispecific antibody 2 comprises a heavy chain variable region having an amino acid sequence as set forth in SEQ ID NO: 6 (PD-1) and a heavy chain variable region having an amino acid sequence as set forth in SEQ ID NO: 14 (LAG-3).

PD-1xLAG-3 bispecific antibody 3 comprises a heavy chain variable region having an amino acid sequence as set forth in SEQ ID NO: 5 (PD-1) and a heavy chain variable region having an amino acid sequence as set forth in SEQ ID NO: 14 (LAG-3).

PD-1xLAG-3 bispecific antibody 4 comprises a heavy chain variable region having an amino acid sequence as set forth in SEQ ID NO: 7 (PD-1) and a heavy chain variable region having an amino acid sequence as set forth in SEQ ID NO: 17 (LAG-3).

PD-1xLAG-3 bispecific antibody 5 comprises a heavy chain variable region having an amino acid sequence as set forth in SEQ ID NO: 8 (PD-1) and a heavy chain variable region having an amino acid sequence as set forth in SEQ ID NO: 17 (LAG-3).

The binding domains of the bispecific antibodies can comprise a CH1 having an amino acid sequence as set forth in SEQ ID NO: 29 The PD-1 binding heavy chain of the bispecific antibodies can comprise a CH2 and CH3 having an amino acid sequence as set forth in SEQ ID NO: 30 and 31, respectively. The LAG-3 binding heavy chain of the bispecific antibodies can comprise a CH2 and CH3 having an amino acid sequence as set forth in SEQ ID NO: 32 and 33, respectively. The PD-1 and LAG-3 binding domains can comprise a light chain variable region having an amino acid sequence as set forth in SEQ ID NO: 24 and a light chain constant region having an amino acid sequence as set forth in SEQ ID NO: 60.

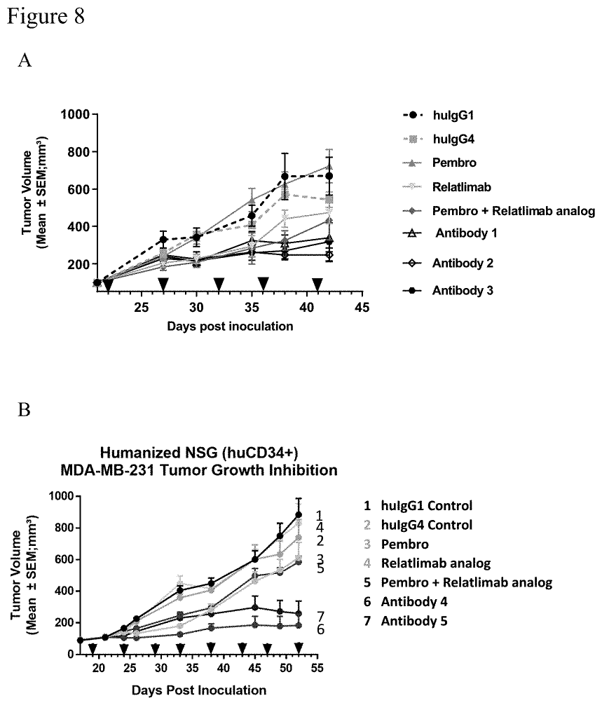

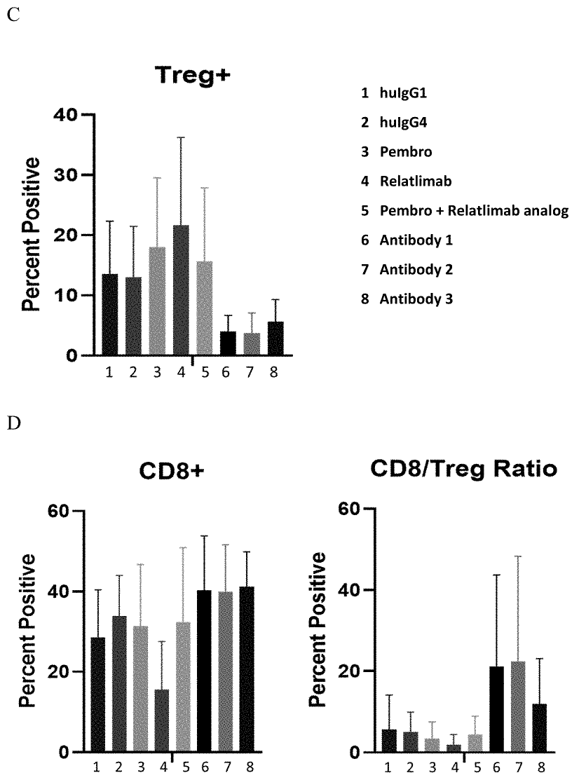

Animals were dosed intraperitoneally every five days for a period of 20 days (FIG. 8A), or every five days for a period of 34 days (FIG. 8B). Tumors were measured using calipers, and the tumor volume was calculated by assimilating them to an ellipsoid using the formula: 1(length)×w² (width)×½. Statistical significance in the efficacy study was determined by One-Way ANOVA. Body weights were also monitored all through the study. Tumors were harvested post treatment, micro-dissected and digested using Tumor Dissociation Kit (Miltenyi Biotec) as per the manufacturer's guidelines, followed by flow cytometry analysis.

### Flow Cytometry Analysis

Upon termination of the in vivo phase, 29 days after start of treatment, tumor cells were analyzed by flow cytometric analysis. To this end, tumors were harvested, transferred into a 15-mL C tube (Miltenyi Biotec) containing 3 mL DMEM medium. To obtain a single cell suspension for flow cytometric analysis, tumors were micro-dissected, and digested using a Tumor Dissociation Kit (Miltenyi Biotec) according to the manufacturer's instructions. Analysis of tumor cells was conducted using Viability dye for live/dead cells and fluorochrome-conjugated antibodies against human CD45 as a leukocyte marker. The flow cytometry panel for this study is as follows:

| Target | Clones | Fluor | Vendor |
|---|---|---|---|
| L/D | N/A | PE-TR | Biolegend |
| mCD45 | 30-F11 | BV605 | BD |
| hCD45 | 2D1 | APC-H7 | BD |
| hCD3 | UCTH1 | BUV395 | BD |
| hCD4 | SK3 | BUV563 | BD |
| hCD8 | RPA-T8 | BUV737 | BD |
| hCD25 | 2A3 | PE-Cy7 | BD |
| hFoxP3 | 236A/E7 | BB700 | BD |
| hTim3 | 7D3 | BV786 | BD |
| hPDl | NAT105 | BV510 | Biolegend |
| hLag3 | 11C3C65 | BV421 | Biolegend |
| hTCFl | 7F11A10 | PE | Biolegend |
| hKi-67 | B56 | BV711 | BD |
| hTox | REA473 | APC | Miltenyi |

Prior to addition of the antibody cocktail, samples were blocked for human and murine Fc receptor for 10 mins at room temperature. Live/dead staining was performed at room temperature for 15 mins prior to addition of the antibody cocktail. Post incubation of the cocktail for 30 mins at room temperature, samples were thoroughly washed and fixed with 2% PFA. Cells were run and analyzed using a BD Symphony Flow Cytometry analyzer and the FlowJo software package.

### Results

Results are shown in FIG. 8. All bispecific antibodies demonstrated better efficacy than the monospecific antibodies as single agent or as combination (FIGS. 8A and B). After 20 days of dosing one study had to be terminated due to increased signs of graft versus host disease in the bispecific antibody treated groups (FIG. 8A), which might be due to increased immune activation upon treatment with the bispecific antibodies. This phenomenon was however not observed among the other groups.

Following takedown of the efficacy study, tumors were dissociated for immune PD analysis by flow cytometry. As mentioned, a 14 color flow cytometry panel was designed and implemented to be evaluate the TILs upon treatment. Evaluating the TILs, all evaluated bispecific antibodies demonstrated decreased percentage of human T cells that are Tregs in the tumors (FIG. 8C). Furthermore, all evaluated bispecific antibodies demonstrated increased ratio of CD8⁺/Tregs in the tumor (FIG. 8D).

### Example 12

### Binding Characteristics

Binding affinity and simultaneous binding of bispecific antibodies was performed by SPR.

The binding affinity of the bispecific antibodies for human PD-1, cynomolgus PD-1, human LAG-3, and cynomolgus LAG-3 was determined in PD-1xLAG-3 bispecific antibody format, and compared with analogs of reference antibodies nivolumab and relatlimab.

Binding affinity was determined in bispecific IgG format using SPR on a BIAcore-T200 instrument using an anti-hulgG antibody immobilized on a CM5 Series S sensor chip. It was also assessed if the two human proteins can be engaged simultaneously by the bispecific antibodies. The binding affinity of bispecific antibodies comprising a PD-1 binding domain comprising a heavy chain variable region having SEQ ID NO: 7, or a PD-1 binding domain comprising a heavy chain variable region having SEQ ID NO: 8, and a LAG-3 binding domain comprising a heavy chain variable region having SEQ ID NO: 17 to human PD-1, cynomolgus PD-1, human LAG-3, and cynomolgus LAG-3 was determined. The binding affinity of the bispecific antibodies was compared with the binding affinity of an analog of reference antibody nivolumab (SEQ ID NO: 21/SEQ ID NO: 22) and of an analog of reference antibody relatlimab (SEQ ID NO: 27/SEQ ID NO: 28). An antibody against an unrelated target was used as a negative control for binding.

Monomeric recombinant antigens used were: huLAG-3 (huLAG-3-His, Sino Biological, Cat. nr. 16498-H08H), cyLAG-3 (cyLAG-3-His, Sino Biological, cat. nr. 90841-C08H), huPD-1 (huPD-1-His, Sino Biological, cat. nr. 10377-H08H) and cyPD-1 (cyPD-1-His, R&D Systems, cat. nr. 8509-PD).

### Immobilization:

Immobilization of goat anti-hulgG Fc (JIR, cat. nr. 109-005-098) on four flow channels of a CMS sensor chip (GE Healthcare; Cat. Nr. BR-1005-30) was performed by amine coupling, using 40 μg/ml of the antibody diluted in 10 mM acetate pH 5.0. The following conditions were used: activation time of 420 seconds, deactivation time of 420 seconds, deactivation buffer: 1 M ethanolamine pH 8.5. A high density of immobilization was achieved, ranging from 9158 to 9428 RU.

### Affinity Determination:

For affinity determination, test and control antibodies were captured by anti-huIgG antibody immobilized on the CM5 sensor chip at a flow rate of 30 μl/min for 60 seconds in only one flow cell. Captured antibody concentration was 20 nM for PD-1 affinity determination and 10 nM for LAG-3 affinity determination. This was followed by a stabilization period of 60 seconds with buffer at a flow rate of 30 μl/min. Five step, two fold, serial dilutions of the antigens were injected, at 30 μl/min, for 60 seconds, in both the flow cell with the captured antibody and a reference flow cell (no captured antibody). Antigen concentrations were 80 nM down to 2.5 nM for huPD-1 and cyPD-1, and 40 to 1.25 nM for hu-LAG-3 and cy-LAG-3. Background correction for buffer effects was performed by injection with buffer alone and the reference flow cell was used for background subtraction.

Following antibody-antigen interaction, an off-rate wash of 300 seconds, at 30 μl/min was done. Regeneration between cycles was done using two 15 μl injections of 10 mM Glycine pH 1.5 at 30 μl/min, followed by a stabilization step of 90 seconds at 90 μl/min. To confirm total regeneration and assay consistency, a repeat run of the reference antibody with all the tested antigen concentrations was performed at the end of the assay and for all antigens tested.

HBS-EP+ buffer was used for PD-1 affinity determination, while, for LAG-3, HBS-EP+ was supplemented with NaCl to a final concentration of 500 mM NaCl, in order to avoid unspecific binding. Results were analyzed in Biacore T200 Evaluation Software. The raw RU signal were blank subtracted (channel with no captured antibody) and background corrected for buffer effects (subtraction of the run with captured antibody but with buffer in the second injection, instead of antigen). 1:1 binding Langmuir fitting was applied to the set of sample curves, using the simultaneous fitting option of the Biacore T200 Evaluation Software to calculate association rate (ka), dissociation rate (kd) and affinity (KD).

The captured bispecific and reference antibodies showed binding to the respective recombinant antigens. No binding of the antigen to the negative control antibody was observed.

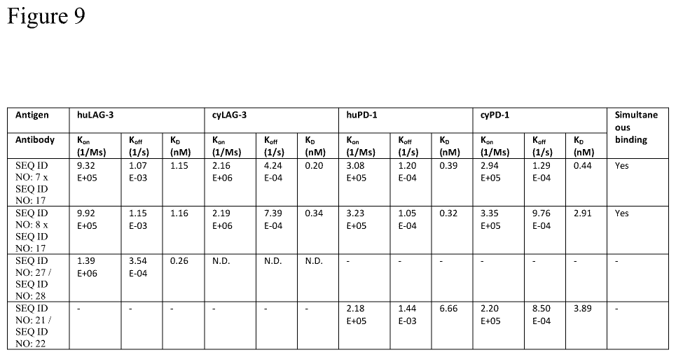

An overview of the data is provided in FIG. 9. The PD-1xLAG-3 bispecific antibodies have a lower affinity for human LAG-3 than the relatlimab analog, and a higher affinity for human and cynomolgus PD-1 than the nivolumab analog. The PD-1xLAG-3 bispecific antibodies bind simultaneously to human PD-1 and human LAG-3.

### Simultaneous Binding:

Simultaneous binding of the bispecific antibodies to hu-LAG-3 and huPD-1 was assayed with a similar set-up as for affinity determination. An immobilized anti-huIgG was used to capture the bispecific antibodies. A mix of nivolumab analog and relatlimab analog reference antibodies was included as a positive control and an antibody against an unrelated target was included as negative control. Then, one of the antigens was injected at a saturating concentration (80 nM for huPD-1 and 40 nM for hu-LAG-3) for 300 sec, to occupy all antigen binding sites. The second antigen was injected sequentially at the same concentration used in injection 1, either alone or in combination with the first antigen (to ensure that all binding sites remained occupied). High salt buffer was used during the whole process, to prevent hu-LAG-3 unspecific binding.

An overview of the data is provided in FIG. 9. The PD-1xLAG-3 bispecific antibodies bind simultaneously to human PD-1 and human LAG-3.

SEQUENCES

SEQ ID NO: 1- Heavy chain variable region - CDRs indicated in bold and underlined
according to Kabat
QVQLQESGPGLVKPSETLSLTCTVSNGSLG [symbol] WIRQPPGRGLEWIG [symbol] [symbol]
RVTMSVDTSKNQFSLNLRSVTAADTAVYYCAR [symbol] W
GQGTLVTVSS
SEQ ID NO: 2- Heavy chain variable region - CDRs indicated in bold and underlined
according to Kabat
QVQLQESGPGLVKPSETLSLTCTVSNGSLG [symbol] WIRQPPGRGLEWIG [symbol] [symbol]
RVTMSVDTSKNQFSLNLRSVTAADTAVYYCAR [symbol] W
GQGTLVTVSS
SEQ ID NO: 3- Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVTTAYLQISSLKAEDTAVYYCAR [symbol] [symbol]
WGQGTLVTVSS
SEQ ID NO: 4- Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVTTAYLQISSLKAEDTAVYYCAR [symbol] [symbol]
WGQGTLVTVSS
SEQ ID NO: 5- Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVTTAYLQISSLKAEDTAVYYCAR [symbol] [symbol]
WGQGTLVTVSS
SEQ ID NO: 6- Heavy chain variable region - CDRs indicated in bold and underlined
according to Kabat
QVQLQESGPGLVKPSETLSLTCTVSDGSIG [symbol] WIRQPPGRGLEWIG [symbol] [symbol]
RVTMSVDTSKNQFSLNLRSVTAADTAVYYCAR [symbol]
WGQGTLVTVSS
SEQ ID NO: 7- Heavy chain variable region - CDRs indicated in bold and underlined
according to Kabat
QVQLQESGPGLVKPSETLSLTCTVSEGSIG [symbol] WIRQPPGRGLEWIG [symbol] [symbol]
RVTMSVDTSKNQFSLNLRSVTAADTAVYYCAR [symbol]
WGQGTLVTVSS
SEQ ID NO: 8- Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVTTAYLQISSLKAEDTAVYYCAR [symbol] [symbol]
WGQGTLVTVSS
SEQ ID NO: 9- Heavy chain variable region - CDRs indicated in bold and underlined
according to Kabat
QVQLQESGPGLVKPSETLSLTCTVSNGSLG [symbol] WIRQPPGRGLEWIG [symbol] [symbol]
RVTMSVDTSKNQFSLNLRSVTAADTAVYYCAR [symbol] W
GQGTLVTVSS
SEQ ID NO: 10 - Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVTTAYLQISSLKAEDTAVYYCAR [symbol] [symbol]
WGQGTLVTVSS
SEQ ID NO: 11 - Heavy chain variable region - CDRs indicated in bold and underlined
according to Kabat IMGT
QVQLQESGPGLVRPSETLSLTCTVSGGSIS [symbol] WIRQPPGKGLEWIG [symbol] [symbol]
RVTISVDTSKTQFSLKLSSVSAADTAVYYCAK [symbol] WG
QGTTVTVSS
SEQ ID NO: 12 - Heavy chain variable region - CDRs indicated in bold and underlined
according to Kabat
EVQLVESGGGVVQPGRSLRLSCAASGFTFS [symbol] WVRQAPGKGLEWVA [symbol] [symbol]
RFTISRDNSKNTLYLQMNSLRAEDTAMYYCAR [symbol] WGQ
GTLVTVSS
SEQ ID NO: 13 - Heavy chain variable region - CDRs indicated in bold and underlined
according to Kabat
EVQLVESGGGVVQPGRSLRLSCAASGFTFS [symbol] WVRQAPGKGLEWVA [symbol] [symbol]
RFTISRDNSKNTLYLQMNSLRAEDTAVYYCAR [symbol] W
GQGTLVTVSS
SEQ ID NO: 14 - Heavy chain variable region - CDRs indicated in bold and underlined
according to Kabat
EVQLVQSGSELKKPGASVKVSCKASGYTFT [symbol] WVRQAPGQGLEWMG [symbol] [symbol]
RFVFSLDTSVSTAYLQIRSLKAEDTAVYYCAR [symbol] W
GQGTLVTVSS
SEQ ID NO: 15 - Heavy chain variable region - CDRs indicated in bold and underlined
according to Kabat
QVQLVQSGAEVKKPGASVKVSCKASGYTFT [symbol] WVRQAPGQGLEWMG [symbol] [symbol]
RVTMTTDTSTSTAYMELRSLRSDDTAVYYCAR [symbol]
WGQGTLVTVSS
SEQ ID NO: 16 - Heavy chain variable region - CDRs indicated in bold and underlined
according to Kabat
QVQLVQSGAEVKKPGASVKVSCKASGYTFT [symbol] WVRQAPGQGLEWMG [symbol] [symbol]
RVTMTTDTSTSTAYMELRSLRSDDTAVYYCAR [symbol] [symbol]
WGQGTLVTVSS
SEQ ID NO: 17 - Heavy chain variable region - CDRs indicated in bold and underlined
according to Kabat
QVQLVQSGSELKKPGASVKVSCKASGYTFT [symbol] WVRQAPGQGLEWMG [symbol] [symbol]
RFVFSLDTSVSTAYLQIRSLKAEDTAVYYCAR [symbol] W
GQGTLVTVSS
SEQ ID NO: 18 - Heavy chain nivolumab analog 1
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCP
APELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 19 - Heavy chain nivolumab analog 2
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
SEQ ID NO: 20 - Heavy chain nivolumab analog 3
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
SEQ ID NO: 21 - Heavy chain nivolumab analog 4
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
SEQ ID NO: 22 - Light chain nivolumab
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 23 - Heavy chain variable region negative control
EVQLVESGGGVVQPGRSLRLSCAASGFTFSNYGMHWVRQAPGKGLEWVAVISYDG
STKYSADSLKGRFTISRDNSKNTLYLQMNSLRADDTAVYYCAKEGWSFDSSGYRSW
FDSWGQGTLVT
SEQ ID NO: 24 - Light chain variable region negative control
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPTFGQGTKVEIK
SEQ ID NO: 25 - Heavy chain motavizumab analog
QVTLRESGPALVKPTQTLTLTCTFSGFSLSTAGMSVGWIRQPPGKALEWLADIWWD
DKKHYNPSLKDRLTISKDTSKNQVVLKVTNMDPADTATYYCARDMIFNFYFDVWG
QGTTVTVSSASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
SEQ ID NO: 26 - Light chain motavizumab analog
DIQMTQSPSTLSASVGDRVTITCSASSRVGYMHWYQQKPGKAPKLLIYDTSKLASGV
PSRFSGSGSGTEFTLTISSLQPDDFATYYCFQGSGYPFTFGGGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 27 - Heavy chain relatlimab analog
QVQLQQWGAGLLKPSETLSLTCAVYGGSFSDYYWNWIRQPPGKGLEWIGEINHRGS
TNSNPSLKSRVTLSLDTSKNQFSLKLRSVTAADTAVYYCAFGYSDYEYNWFDPWGQ
GTLVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
SEQ ID NO: 28 - Light chain relatlimab analog
EIVLTQSPATLSLSPGERATLSCRASQSISSYLAWYQQKPGQAPRLLIYDASNRATGIP
ARFSGSGSGTDFTLTISSLEPEDFAVYYCQQRSNWPLTFGQGTNLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 29 - CH1
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRV
SEQ ID NO: 30 - CH2
APELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
SEQ ID NO: 31 -CH3
GQPREPQVYTDPPSREEMTKNQVSLTCEVKGFYPSDIAVEWESNGQPENNYKTTPPV
LDSDGSFFLYSKLTVDKSRWQQGNVFSCSVMHEALHNHYTQKSLSLSPGK
SEQ ID NO: 32 - CH2
APELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
SEQ ID NO: 33 - CH3
GQPREPQVYTKPPSREEMTKNQVSLKCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 34 - Nivolumab analog heavy chain variable region
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSS
SEQ ID NO: 35 - Nivolumab analog light chain variable region
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIK
SEQ ID NO: 36 - HCDR1 according to Kabat
FDFWS
SEQ ID NO: 37 - HCDR2 according to Kabat
YIYYSGSWSLNPSFKG
SEQ ID NO: 38 - HCDR3 according to Kabat
GGYTGYGGDWFDP
SEQ ID NO: 39 - HCDR1 according to Kabat
FEFWS
SEQ ID NO: 40 - HCDR2 according to Kabat
YIVYSGSHSVSPSLKT
SEQ ID NO: 41 - HCDR3 according to Kabat
GGYTGHGGDWFDT
SEQ ID NO: 42 - HCDR1 according to Kabat
RFALS
SEQ ID NO: 43 - HCDR2 according to Kabat
WIDPNTGTPTYAQDFTG
SEQ ID NO: 44 - HCDR3 according to Kabat
SLGYCGSDICYPNGILDN
SEQ ID NO: 45 - HCDR1 according to Kabat
RFAVN
SEQ ID NO: 46 - HCDR2 according to Kabat
WIDPNTGTPTYAQGVTN
SEQ ID NO: 47 - HCDR3 according to Kabat
SLGYCSSDICYPNLIFDN
SEQ ID NO: 48 - HCDR1 according to Kabat
RFALH
SEQ ID NO: 49 - HCDR2 according to Kabat
WIDPNTGTPTFAQGVTG
SEQ ID NO: 50 - HCDR3 according to Kabat
SLGYCDSDICYPNWIFDN
SEQ ID NO: 51 - HCDR1 according to Kabat
YHFWS
SEQ ID NO: 52 - HCDR2 according to Kabat
YIVYSGSYNVNPSLKT
SEQ ID NO: 53 - HCDR3 according to Kabat
GGYTGYGGDWFDP
SEQ ID NO: 54 - HCDR1 according to Kabat
YHFWS
SEQ ID NO: 55 - HCDR2 according to Kabat
YIVYSGSYNVNPSLKT
SEQ ID NO: 56 - HCDR3 according to Kabat
GGYTGYGGDWFDP
SEQ ID NO: 57 - HCDR1 according to Kabat
RFALH
SEQ ID NO: 58 - HCDR2 according to Kabat
WIDPNTGTPTFAQGVTG
SEQ ID NO: 59 - HCDR3 according to Kabat
SLGYCDSDICYPNWIFDN
SEQ ID NO: 60 LCDR1 according to IMGT
QSISSY
SEQ ID NO: 61 LCDR2 according to IMGT
AAS
SEQ ID NO: 62 LCDR3 according to IMGT
QQSYSTPPT
SEQ ID NO: 63 Light chain variable region - CDRs indicated in bold and
underlined according to IMGT
DIQMTQSPSSLSASVGDRVTITCRAS [symbol] LNWYQQKPGKAPKLLIY [symbol] SLQSGV
PSRFSGSGSGTDFTLTISSLQPEDFATYYC [symbol] FGQGTRLEIK
SEQ ID NO: 64 Light chain variable region - CDRs indicated in bold and
underlined according to IMGT
EIVMTQSPATLSVSPGERATLSCRAS [symbol] LAWYQOQKPGQAPRLLIY [symbol] TRATGI
PARFSGSGSGTEFTLTISSLQSEDFAVYYC [symbol] FGQGTKVEIK
SEQ ID NO: 65 Light chain variable region - CDRs indicated in bold and
underlined according to IMGT
EIVLTQSPGTLSLSPGERATLSCRAS [symbol] LAWYQQKPGQAPRLLIY [symbol] SRATGI
PDRFSGSGSGTDFTLTISRLEPEDFAVYYC [symbol] FGQGTKVEIK
SEQ ID NO: 66 Light chain variable region - CDRs indicated in bold and
underlined according to IMGT
SYVLTQPPSVSVAPGETARITCGGD [symbol] VYWYQQKSGQAPVLVIY [symbol] DRPSGI
PERFSGSNSGNTATLTISRVEAGDEADYYC [symbol] FGGGTKLTVL
SEQ ID NO: 67 V region
DIQMTQSPSSLSASVGDRVTITCRASQSISSYLNWYQQKPGKAPKLLIYAASSLQSGV
PSRFSGSGSGTDFTLTISSLQPEDFATYYCQQSYSTP
SEQ ID NO: 68 V region
EIVMTQSPATLSVSPGERATLSCRASQSVSSNLAWYQQKPGQAPRLLIYGASTRATGI
PARFSGSGSGTEFTLTISSLQSEDFAVYYCQQYNNWP
SEQ ID NO: 69 V region
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSP
SEQ ID NO: 70 V region
SYVLTQPPSVSVAPGETARITCGGDNIGRKSVYWYQQKSGQAPVLVIYYDSDRPSGIP
ERFSGSNSGNTATLTISRVEAGDEADYYCQVWDGSSDH
SEQ ID NO: 71 Light chain constant region
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 72 CH2
APELLGGPSVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTISKAK
SEQ ID NO: 73 CH3
GQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 74 - HCDR1 according to Kabat
SYSWS
SEQ ID NO: 75 - HCDR2 according to Kabat
YIDYSGSTNYNPSLKS
SEQ ID NO: 76 - HCDR3 according to Kabat
DLLYKWNYVEGFDI
SEQ ID NO: 77 - HCDR1 according to Kabat
SYDTH
SEQ ID NO: 78 - HCDR2 according to Kabat
VISYDGSNKYYADSVKG
SEQ ID NO: 79 - HCDR3 according to Kabat
ERGWDVFDI
SEQ ID NO: 80 - HCDR1 according to Kabat
SYGMH
SEQ ID NO: 81 - HCDR2 according to Kabat
VISYHGSDKYYADSVKG
SEQ ID NO: 82 - HCDR3 according to Kabat
DGDNWDVFDI
SEQ ID NO: 83 - HCDR1 according to Kabat
TNALN
SEQ ID NO: 84 - HCDR2 according to Kabat
WINTHTGNPTYAQGFIG
SEQ ID NO: 85 - HCDR3 according to Kabat
EPNWGVYFDY
SEQ ID NO: 86 - HCDR1 according to Kabat
SYGIS
SEQ ID NO: 87 - HCDR2 according to Kabat
WISAYSGNTNYAQKLQG
SEQ ID NO: 88 - HCDR3 according to Kabat
DGSGWDDFDY
SEQ ID NO: 89 - HCDR1 according to Kabat
SYGIS
SEQ ID NO: 90 - HCDR2 according to Kabat
WISAYSGNTNYAQKLQG
SEQ ID NO: 91 - HCDR3 according to Kabat
GSILAAQMWGDI
SEQ ID NO: 92 - HCDR1 according to Kabat
TNALN
SEQ ID NO: 93 - HCDR2 according to Kabat
WINTHTGNPTYAQGFIG
SEQ ID NO: 94 - HCDR3 according to Kabat
EPNWGVYFDY

1. A multispecific binding moiety comprising an anti-human PD-1 binding domain, wherein (i) the anti-human PD-1 binding domain has higher binding affinity for human PD-1 than a reference anti-human PD-1 binding domain, wherein the reference anti-human PD-1 binding domain comprises a heavy chain variable region having an amino acid sequence as set forth in SEQ ID NO: 34 and a light chain variable region having an amino acid sequence as set forth in SEQ ID NO: 35, in particular wherein the binding affinity is measured by surface plasmon resonance, or (ii) the anti-human PD-1 binding domain provides comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 34 and two light chain variable regions having an amino acid sequence as set forth in SEQ ID NO: 35, in particular wherein the potency in blocking ligand binding to PD-1 is measured in a PD-1/PD-L1 or PD-1/LAG-3 reporter assay.

2. (canceled)

3. The multispecific binding moiety according to claim 1, wherein the anti-human PD-1 binding domain comprises at least a heavy chain variable region and a light chain variable region, and wherein the light chain variable region preferably is a light chain variable region of a light chain that is capable of pairing with multiple heavy chains having different epitope specificities.

4. (canceled)

5. The multispecific binding moiety according to claim 1, wherein the anti-human PD-1 binding domain has at least a ten-fold higher binding affinity for human PD-1 than the reference anti-human PD-1 binding domain or wherein the anti-human PD-1 binding domain has a ten-fold higher binding affinity for human PD-1 than the reference anti-human PD-1 binding domain.

6. (canceled)

7. The multispecific binding moiety according to claim 1, wherein the anti-human PD-1 binding domain has a binding affinity for human PD-1 in a range of about 0.1-1.0 nM, in particular in a range of about 0.3-0.8 nM, more in particular in a range of about 0.38-0.78 nM.

8. The multispecific binding moiety according to claim 1, wherein the binding affinity is measured with both the anti-human PD-1 binding domain and the reference anti-human PD-1 binding domain in a bivalent monospecific IgG format, or wherein the binding affinity is measured with the anti-human PD-1 binding domain in a bivalent bispecific IgG format and the reference anti-human PD-1 binding domain in a bivalent monospecific IgG format.

9. (canceled)

10. (canceled)

11. The multispecific binding moiety according to claim 1, wherein a comparable potency in blocking ligand binding to PD-1 is a potency within a 5 fold range of the potency in blocking ligand binding to PD-1 of the reference anti-human PD-1 antibody, including a 5, 4, 3, and 2 fold, deviation from the potency in blocking ligand binding to PD-1 of the reference anti-human PD-1 antibody.

12-14. (canceled)

15. A multispecific binding moiety comprising an anti-human PD-1 binding domain, wherein the anti-human PD-1 binding domain comprises a heavy chain variable region, wherein the heavy chain variable region comprises a heavy chain CDR1 (HCDR1) from a heavy chain variable region having an amino acid sequence from the group consisting of SEQ ID NOS: 1-8, a heavy chain CDR2 (HCDR2) from a heavy chain variable region having an amino acid sequence from the group consisting of SEQ ID NOS: 1-8, and a heavy chain CDR3 (HCDR3) from a heavy chain variable regions having an amino acid sequence from the group consisting of SEQ ID NOS: 1-8, in particular wherein the heavy chain variable region comprises:
a) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
b) heavy chain CDR1 (HCDR1), heavy chain CDR2 (HCDR2), and heavy chain CDR3 (HCDR3), having an amino acid sequence as set forth in SEQ ID NO: 39, SEQ ID NO: 40, and SEQ ID NO: 41, respectively;
c) heavy chain CDR1 (HCDR1), heavy chain CDR2 (HCDR2), and heavy chain CDR3 (HCDR3), having an amino acid sequence as set forth in SEQ ID NO: 42, SEQ ID NO: 43, and SEQ ID NO: 44, respectively;
d) heavy chain CDR1 (HCDR1), heavy chain CDR2 (HCDR2), and heavy chain CDR3 (HCDR3), having an amino acid sequence as set forth in SEQ ID NO: 45, SEQ ID NO: 46, and SEQ ID NO: 47, respectively;
e) heavy chain CDR1 (HCDR1), heavy chain CDR2 (HCDR2), and heavy chain CDR3 (HCDR3), having an amino acid sequence as set forth in SEQ ID NO: 48, SEQ ID NO: 49, and SEQ ID NO: 50, respectively;
f) heavy chain CDR1 (HCDR1), heavy chain CDR2 (HCDR2), and heavy chain CDR3 (HCDR3), having an amino acid sequence as set forth in SEQ ID NO: 51, SEQ ID NO: 52, and SEQ ID NO: 53, respectively;
g) heavy chain CDR1 (HCDR1), heavy chain CDR2 (HCDR2), and heavy chain CDR3 (HCDR3), having an amino acid sequence as set forth in SEQ ID NO: 54, SEQ ID NO: 55, and SEQ ID NO: 56, respectively; or
h) heavy chain CDR1 (HCDR1), heavy chain CDR2 (HCDR2), and heavy chain CDR3 (HCDR3), having an amino acid sequence as set forth in SEQ ID NO: 57, SEQ ID NO: 58, and SEQ ID NO: 59, respectively;
wherein each of the HCDRs may comprise at most three, two, or one amino acid substitutions.

16. (canceled)

17. The multispecific binding moiety according to claim 15, comprising a heavy chain variable region having an amino acid sequence as set forth in any one of SEQ ID NO: 1-8, or having at least 80%, preferably 85%, more preferably 90%, or most preferably 95% sequence identity thereto.

18. (canceled)

19. The multispecific binding moiety according to claim 15, further comprising a binding domain that binds to a cell surface moiety expressed on an immune effector cell, in particular further comprising an anti-human LAG-3 binding domain.

20. (canceled)

21. The multispecific binding moiety according to claim 19, wherein the anti-human LAG-3 binding domain comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 11;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 12;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 13;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 14;
e) heavy chain CDR1 (HCDR1), heavy chain CDR2 (HCDR2), and heavy chain CDR3 (HCDR3), of a heavy chain variable region having an amino acid sequence as set forth in SEQ ID NO: 15;
f) heavy chain CDR1 (HCDR1), heavy chain CDR2 (HCDR2), and heavy chain CDR3 (HCDR3), of a heavy chain variable region having an amino acid sequence as set forth in SEQ ID NO: 16; or
g) heavy chain CDR1 (HCDR1), heavy chain CDR2 (HCDR2), and heavy chain CDR3 (HCDR3), of a heavy chain variable region having an amino acid sequence as set forth in SEQ ID NO: 17,
wherein each of the HCDRs may comprise at most three, two, or one amino acid substitutions.

22. The multispecific binding moiety according to claim 19, wherein the anti-human LAG-3 binding domain comprises a heavy chain variable region having an amino acid sequence as set forth in any one of SEQ ID NOS: 11-17, or having at least 80%, preferably 85%, more preferably 90%, or most preferably 95% sequence identity thereto.

23. The multispecific binding moiety according to claim 19, wherein the anti-human LAG-3 binding domain further comprises a light chain variable region, preferably a light chain variable region of a light chain that is capable of pairing with multiple heavy chains having different epitope specificities, in particular the same light chain variable region as that of the anti-human PD-1 binding domain.

24-27. (canceled)

28. The multispecific binding moiety according to claim 1, wherein the binding moiety is monovalent for binding to human PD-1.

29. A pharmaceutical composition comprising an effective amount of the multispecific binding moiety according to claim 1, and a pharmaceutically acceptable carrier.

30-32. (canceled)

33. A method for treating a disease, comprising administering an effective amount of a multispecific binding moiety as claimed in claim 1 to an individual in need thereof.

34. A method for treating a disease associated with a suppressed immune system, comprising administering an effective amount of a multispecific binding moiety as claimed in claim 1 to an individual in need thereof.

35. A method for treating cancer, comprising administering an effective amount of a multispecific binding moiety as claimed in claim 1 to an individual in need thereof.

36. A vector comprising a nucleic acid sequence encoding the heavy chain variable region of an anti-human PD-1 binding domain as defined in claim 1 and a nucleic acid sequence encoding the heavy chain variable region of an anti-human LAG-3 binding domain, wherein the anti-human LAG-3 binding domain comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 11;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 12;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 13;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 14;
e) heavy chain CDR1 (HCDR1), heavy chain CDR2 (HCDR2), and heavy chain CDR3 (HCDR3), of a heavy chain variable region having an amino acid sequence as set forth in SEQ ID NO: 15;
f) heavy chain CDR1 (HCDR1), heavy chain CDR2 (HCDR2), and heavy chain CDR3 (HCDR3), of a heavy chain variable region having an amino acid sequence as set forth in SEQ ID NO: 16; or
g) heavy chain CDR1 (HCDR1), heavy chain CDR2 (HCDR2), and heavy chain CDR3 (HCDR3), of a heavy chain variable region having an amino acid sequence as set forth in SEQ ID NO: 17,
wherein each of the HCDRs may comprise at most three, two, or one amino acid substitutions as defined in claim 21 or 22.

37. The vector according to claim 36, wherein the vector further comprises a nucleic acid sequence encoding a CH1 region and preferably a hinge, CH2 and CH3 region.

38. The vector according to claim 36, wherein the vector further comprises at least one nucleic acid sequence encoding a light chain variable region, and preferably a CL region, in particular wherein the light chain variable region is a light chain variable region of a light chain that is capable of pairing with multiple heavy chains having different epitope specificities.

39. (canceled)

40. A cell comprising a nucleic acid sequence encoding the heavy chain variable region of an anti-human PD-1 binding domain as defined in claim 1 and a nucleic acid sequence encoding the heavy chain variable region of an anti-human LAG-3 binding domain, wherein the anti-human LAG-3 binding domain comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 11;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 12;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 13;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 14;
e) heavy chain CDR1 (HCDR1), heavy chain CDR2 (HCDR2), and heavy chain CDR3 (HCDR3), of a heavy chain variable region having an amino acid sequence as set forth in SEQ ID NO: 15;
f) heavy chain CDR1 (HCDR1), heavy chain CDR2 (HCDR2), and heavy chain CDR3 (HCDR3), of a heavy chain variable region having an amino acid sequence as set forth in SEQ ID NO: 16; or
g) heavy chain CDR1 (HCDR1), heavy chain CDR2 (HCDR2), and heavy chain CDR3 (HCDR3), of a heavy chain variable region having an amino acid sequence as set forth in SEQ ID NO: 17,
wherein each of the HCDRs may comprise at most three, two, or one amino acid substitutions.

41. The cell according to claim 40, wherein the cell further comprises a nucleic acid sequence encoding a CH1 region and preferably a hinge, CH2 and CH3 region.

42. The cell according to claim 40, wherein the cell further comprises at least one nucleic acid sequence encoding a light chain variable region, and preferably a CL region.

43. A cell producing a multi specific binding moiety, wherein the cell is a recombinant cell transformed with the vector as claimed in claim 36.

44. (canceled)
